(19) United States
(12) Patent Application Publication
(10) Pub. No.: US 2011/0124841 A1
(43) Pub. Date: May 26, 2011

(54) Polypeptides Modified by Protein Trans-Splicing Technology

(76) Inventors: James F. Monthony, Prince Edward Island (CA); Paul Xiang-Qin Liu, Nova Scotia (CA); Li Yang, Prince Edward Island (CA); Kaisong Zhou, Nova Scotia (CA)

(21) Appl. No.: 12/747,653

(22) PCT Filed: Dec. 12, 2008
(86) PCT No.: PCT/CA08/02171

§ 371 Date: Sep. 7, 2010

Related U.S. Application Data
(60) Provisional application No. 61/013,426, filed on Dec. 13, 2007.

(51) Int. Cl. C07K 14/00; C07K 1/107; C07K 2/00; C07H 21/00; C12N 15/63; C12N 1/21; C12P 21/02

(52) U.S. Cl. 530/324; 530/345; 530/300; 536/23.4; 435/320.1; 435/252.33; 435/69.7; 530/350; 536/23.1; 435/69.1

(57) ABSTRACT

The present invention relates to a method of preparing modified polypeptides, by linking a target polypeptide to a carrier molecule that is designed to bear one or more water-soluble polymer molecules, via protein trans-splicing. The polymer molecules can be attached to the carrier molecule either before or after ligation to the target polypeptide. Novel protein trans-splicing elements (known as “split inteins”) and trans-splicing partners are also provided.

### FIELD OF THE INVENTION

The present invention relates generally to the field of protein modification. More particularly, the present invention relates to a method of preparing modified polypeptides, by linking a target polypeptide to a carrier molecule that bears one or more water-soluble polymer molecules (such as poly(ethylene glycol) and the like), via protein trans-splicing (PTS). Novel split inteins and splicing partners for use in the PTS-based method are also provided.

### BACKGROUND OF THE INVENTION

Conjugation of water-soluble polymers to therapeutic polypeptides is a well-established drug-enhancement strategy. Poly(ethylene glycol) (PEG) is often used for this purpose, in a process that is commonly referred to as “PEGylation”. PEGylation can produce alterations in the physiochemical properties of polypeptides including changes in conformation, electrostatic binding, hydrophobicity, etc. These physical and chemical changes can increase systemic retention of therapeutic polypeptides. In addition, PEGylation can influence the binding affinity of the therapeutic polypeptide to cell receptors and can alter absorption and distribution patterns. Thus, PEGylated polypeptides can have significant pharmacological advantages over the corresponding un-PEGylated form, such as: improved drug solubility; extended circulating life; increased drug stability; enhanced protection from proteolytic degradation; reduced immunogenicity. PEGylated polypeptides also provide opportunities for new delivery formats and dosing regimens, e.g. reduced dosage frequency, without diminished efficacy and/or with potentially reduced toxicity.

One of the key challenges in this field is specificity. In many cases, it is desirable to modify a polypeptide once, at a single site. However, polypeptides often have multiple copies of the targeted amino acid residue, which commonly results in product mixtures when conventional PEGylation methods are employed.

Another challenge in this field relates to PEGylation at the carboxy-terminal end of a protein.

To some extent, protocols have been developed to address these problems. For example, some methods have been devised to specifically PEGylate the amino-terminus of a target polypeptide (see for example U.S. Pat. No. 6,077,939; U.S. Pat. No. 7,090,835; U.S. Pat. No. 5,621,039; and Gilmore J M, Scheck R A, Esser-Kahn A P, Joshi N S, Francis M B. “N-terminal protein modification through a biomimetic transamination reaction.” Angew Chem Int Ed Engl. (2006) 45(32):5307-11.). Other methods have been devised that introduce an unpaired cysteine residue into a target polypeptide, to serve as a specific site for PEGylation at the carboxy-terminus or other positions in the target peptide (see for example U.S. Pat. No. 7,214,779; and Doherty D H, et al. “Site-specific PEGylation of Engineered Cysteine Analogues of Recombinant Human Granulocyte-Macrophage Colony-Sytimluating Factor.” Bioconjugate Chem. (2005) 16, 1291-1298). Other methods have been devised that introduce an unnatural amino acid residue into a target polypeptide, to serve as a specific site for PEGylation (see for example U.S. Pat. No. 7,230,068).

However, all of these PEGylation procedures have limitations. For example, these processes may produce the desired PEGylated polypeptide in low yield, or reduce the bioactivity of the therapeutic polypeptide (e.g. due to unfolding), or are labor-intensive and involve many steps, etc. Often, side-reactions still occur to some extent, thereby resulting in some degree of unwanted side-products and providing a mixture of products that can have variable bioactive properties and can be difficult or expensive to resolve. In addition, in order to utilize some of these processes, it may be necessary to mutate the therapeutic polypeptide to introduce a suitable target residue; this approach can be problematic, as mutations may alter the bioactivity of the therapeutic polypeptide (e.g. due to changes in secondary structure, or dimerization due to unpaired cysteine residues, etc.), and the ensuing PEGylation reaction may still produce unwanted side-products. In the result, conventional PEGylation procedures are often inefficient and/or wasteful of the therapeutic polypeptide starting materials.

Therefore, there remains a need for alternative methods for attaching water-soluble polymers to polypeptides.

### SUMMARY OF THE INVENTION

The present invention provides a method of preparing modified polypeptides that are conjugated to one or more water-soluble polymer molecules via a carrier molecule. The method utilizes protein trans-splicing (PTS) technology to link a target polypeptide to a carrier molecule component that is designed to carry one or more water-soluble polymer molecules, such as poly(ethylene glycol) (PEG), poly(ethyleneglycol) monomethyl ether (MPEG) and the like. The water-soluble polymer molecule(s) can be attached to the carrier molecule either before or after it is ligated to the therapeutic polypeptide. Also provided are novel polypeptides that find utility for example in the PTS-based method of the invention.

Thus, in a first aspect, the present invention provides a method of modifying a target polypeptide, comprising: (a) providing a first trans-splicing partner which comprises a first component of a split intein in operative linkage with a first extein segment, wherein the first extein comprises at least one functional group suitable for attaching at least one water-soluble polymer molecule; (b) providing a second trans-splicing partner which comprises a second component of the split intein in operative linkage with a second extein segment that comprises the target polypeptide, wherein said first and second trans-splicing partners are capable of cooperating to provide protein trans-splicing (PTS) activity; and (c) contacting said first and second trans-splicing partners under conditions suitable to induce excision of the first and second components of the split intein and joining of the extein segments, so as to ligate the first extein to the second extein; wherein at least one water-soluble polymer is attached to the first extein either before or after the first extein is ligated to the second extein.

In embodiments, a polypeptide of interest can be split to provide the target polypeptide and a carrier molecule for attaching a water-soluble polymer molecule (or the exteins comprising them).

In embodiments, the polymer molecule can be attached to the first extein prior to ligating it to the second extein, to produce a product in which polymer is attached to only the first extein and/or to protect the second extein from the chemical conditions used to attach polymer to the first extein.

Some of the polypeptides produced by the above-described method are believed to be novel, due to incorporation of novel first exteins comprising amino acid sequences such as those set fort in SEQ ID NOs:11, 12, 16 or 17. Thus, in a further aspect, the present invention provides a chemically modified polypeptide produced by the method described above, wherein the chemically modified polypeptide comprises an amino acid sequence as set forth in SEQ ID NOs:11, 12, 16, or 17.

Some of the polypeptides that are useful in the above-described method are also believed to be novel. Thus, in a further aspect, the present invention provides a polypeptide comprising: (a) an N-terminal or C-terminal component of a split intein; and (b) an extein segment that comprises at least one functional group suitable for attaching at least one water-soluble polymer molecule, wherein the extein segment is in operative linkage with the split intein component; or a conjugate thereof which is covalently bonded to said water-soluble polymer. In embodiments, the polypeptide, or the conjugate thereof, comprises amino acid residues 388 to 453 of SEQ ID NO:1, 398 to 453 of SEQ ID NO:1, 398 to 449 of SEQ ID NO:3, or 388 to 449 of SEQ ID NO:3. The present invention further provides nucleic acid molecules encoding such polypeptides, expression vectors comprising such nucleic acid molecules, host cells comprising such expression vectors, and methods for preparing the polypeptides described above by culturing such host cells. The invention further provides a kit comprising such polypeptides, or a conjugate thereof, together with instructions for use in chemically modifying a target polypeptide.

Some of the components of split inteins disclosed herein are also believed to be novel. Thus, in a further aspect, the present invention provides a polypeptide comprising a component of a split intein, wherein the polypeptide comprises the amino acid sequence as set forth in SEQ ID NO: 6, SEQ ID NO:9, SEQ ID NO:8, or SEQ ID NO:10, or a variant thereof having at least 50% identity thereto and that is capable of interacting with a complementary component of a split intein to provide trans-splicing activity. The present invention further provides nucleic acid molecules encoding such polypeptides, expression vectors comprising such nucleic acid molecules, host cells comprising such expression vectors, and methods for preparing the polypeptides described above by culturing such host cells. The invention further provides the use of such polypeptides in protein trans-splicing reactions.

The invention further provides a kit comprising: (a) a polypeptide comprising a (i) an N-terminal or C-terminal component of a split intein; and (ii) an extein segment that comprises a carrier molecule, wherein said carrier molecule has at least one functional group suitable for attaching at least one water-soluble polymer molecule, and wherein the extein segment is in operative linkage with the split intein component; or a conjugate thereof which is covalently bonded to said water-soluble polymer; and (b) instructions for use to splice said extein segment, or conjugate thereof, to a target polypeptide. The kit may further comprise an expression vector comprising a second component of the split intein segment and restriction sites for inserting a DNA molecule encoding a target polypeptide of interest in operative linkage with the second split intein component.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1A: Principle of protein trans-splicing (PTS). The two halves of the split intein, labeled as I_{N} and l_{C}, associate and fold to form a functional intein. This functional intein can then undergo a pseudo-intramolecular protein splicing reaction, wherein the flanking polypeptides, termed the N-extein (E_{N}) and C-extein (E_{C}), are ligated together and the intein excises itself.

FIG. 1B: Schematic illustration of constructs of the Recombinant Proteins made in Examples 1 to 4 (described below). MBP: maltose binding protein sequence. H:His-tag sequence (six (6) consecutive histidines). E_{C} is the C-extein, which is a cysteine-containing 7-aa peptide sequence. I_{C} is the C-intein and I_{N} is the N-intein of the engineered SB split intein components and the engineered SG split intein components of these proteins.

FIG. 2: Engineered split intein sequences compared to their native intein sequences. Sequences of inteins segments are shown in upper case letters, with flanking extein residues shown in lower case letters. SBnative is the native Ssp DnaB intein (SEQ ID NO:5). SBsplit I_{N} is the engineered split intein N-terminal component (residues 2 to 103 of SEQ ID NO:6, or residues 397 to 498 of SEQ ID NO:2) and SBsplit I_{C} is the engineered split intein C-terminal component (residues 1 to 49 of SEQ ID NO:9, or residues 398 to 446 of SEQ ID NO:1) used respectively in constructing the SB N-protein (SEQ ID NO:2) and the SB C-protein precursor (SEQ ID NO:1). SGnative (SEQ ID NO:7) is the native Ssp GyrB intein. SGsplit I_{N} is the engineered split intein N-terminal component (residues 2 to 112 of SEQ ID NO:8, or residues 394 to 504 of SEQ ID NO:4) and SGsplit I_{C} is the engineered split intein C-terminal component (residues 1 to 45 of SEQ ID NO:10, or residues 398 to 442 of SEQ ID NO:3) used in the SG N-protein (SEQ ID NO:4) and the SG C-protein precursor (SEQ ID NO:3), respectively.

FIG. 3: PEGylation of the SB and SG C-protein precursors. Top, schematic illustration of the PEGylation. I_{C} is the split intein C-terminal component in the C-proteins. PEG: an activated polyethylene glycol. Other symbols are same as in FIG. 1. Bottom, SDS-PAGE analysis of the PEGylation. Lane 1, the SB C-protein precursor before PEGylation. Lane 2, the SB C-protein precursor (SEQ ID NO:1) after PEGylation. Lane 3, the SG C-protein precursor before PEGylation. Lane 4, the SG C-protein precursor (SEQ ID NO:3) after PEGylation.

FIG. 4: Cleavage of PEGylated C-protein precursors to provide PEGylated C-proteins. Top, schematic illustration of the cleavage. Symbols are same as in FIGS. 1 and 3. The Factor Xa protease cleavage site is as marked. Bottom, SDS-PAGE analysis of the cleavages. Lane 1, the PEGylated SB C-protein precursor (SEQ ID NO:1) before cleavage. Lane 2, the PEGylated SB C-protein precursor after cleavage. Lane 3, the PEGylated SG C-protein precursor (SEQ ID NO:3) before cleavage. Lane 4, the PEGylated SG C-protein precursor after cleavage. The respective cleavage products are SB C-protein (residues 388 to 453 of SEQ ID NO:1) and SG C-protein (residues 388 to 449 of SEQ ID NO:3). The dotted arrow marks the expected position for the small PEGylated cleavage product that was not visualized by Coomassie blue staining.

FIG. 5: Trans-splicing of the PEGylated C-protein with the N-protein. Top, schematic illustration of the trans-splicing. Symbols are same as in FIGS. 1, 3, and 4. Middle and bottom, SDS-PAGE analysis of the trans-splicing reaction, with the protein bands of interest marked by arrows and symbols, including the dotted arrow marking the expected position for the small PEGylated C-protein that could not be visualized by Coomassie blue staining. Lanes 1 and 2: the SB N-protein (SEQ ID NO:2) and the partially purified PEGylated SB C-protein (residues 388 to 453 of SEQ ID NO:1), respectively. Lanes 3 and 4: mixture (approximately 1:1) of the SB N-protein and the PEGylated SB C-protein, after incubation at 4° C. and at room temperature, respectively. Lanes 6 and 7: the SG N-protein (SEQ ID NO:4) and the partially purified PEGylated SG C-protein (residues 388 to 449 of SEQ ID NO:3), respectively. Lanes 8 and 9: mixture (approximately 1:1) of the SG N-protein and the PEGylated SG C-protein, after incubation at 4° C. and at room temperature for trans-splicing, respectively. Lane 5, hybrid mixture of the SG N-protein and the PEGylated SB C-protein, after incubation at room temperature. Lane 10, hybrid mixture of the SB N-protein and the PEGylated SG C-protein, after incubation at room temperature. Lanes 11 and 12: the SG N-protein and the partially purified PEGylated SG C-protein, respectively. Lanes 13: mixture (approximately 1:5) of the SG N-protein and the PEGylated SG C-protein, after incubation at room temperature for trans-splicing.

FIGS. 6A and 6B: Nucleic acid sequence (SEQ ID NO:22) and deduced amino acid sequence (SEQ ID NO:1) of SB C-protein precursor comprising the following segments: maltose binding protein (MBP), Factor Xa protease cleavage site, histidine tag (H), SB split C-intein (I_{C}) and C-extein (E_{C}) which comprise a PEGylation site (Cys). (SEQ ID NO:1).

FIGS. 7A and 7B: Nucleic acid sequence (SEQ ID NO:23) and deduced amino acid sequence (SEQ ID NO:2) of SB N-protein comprising the following segments: maltose binding protein (MBP) and SB split N-intein (I_{N}). (SEQ ID NO:2).

FIGS. 8a and 8B: Nucleic acid sequence (SEQ ID NO:24) and deduced amino acid sequence(SEQ ID NO:3) of SG C-protein precursor comprising the following segments: maltose binding protein (MBP), Factor Xa cleavage site, histidine tag (H), SG split C-intein (I_{C}) and C-extein (E_{C}) which comprise a PEGylation site (Cys).

FIGS. 9A and 9B: Nucleic acid sequence (SEQ ID NO:25) and deduced amino acid sequence (SEQ ID NO:4) of SG N-protein comprising the following segments: maltose binding protein (MBP) and SG split N-intein (I_{N}).

FIG. 10: Product of SG N-protein and SG C-protein trans-splicing reaction: amino acid sequence shown. SEQ ID NO:19

FIG. 11: Product of SB N-protein and SB C-protein trans-splicing reaction: amino acid sequence shown. SEQ ID NO:20

### DETAILED DESCRIPTION

Generally, the present invention relates to a method of preparing modified polypeptides that are conjugated to one or more water-soluble polymer molecules via a carrier molecule. The method utilizes protein trans-splicing (PTS) technology to link a target polypeptide to a carrier molecule component that is designed to carry one or more water-soluble polymer molecules polymers, such as poly(ethylene glycol) (PEG), poly(ethylene glycol) monomethyl ether (MPEG) and the like. The water-soluble polymer molecule(s) can be attached to the carrier molecule either before or after it is ligated to the therapeutic polypeptide. Also provided are novel polypeptides that find utility for example in the PTS-based method of the invention.

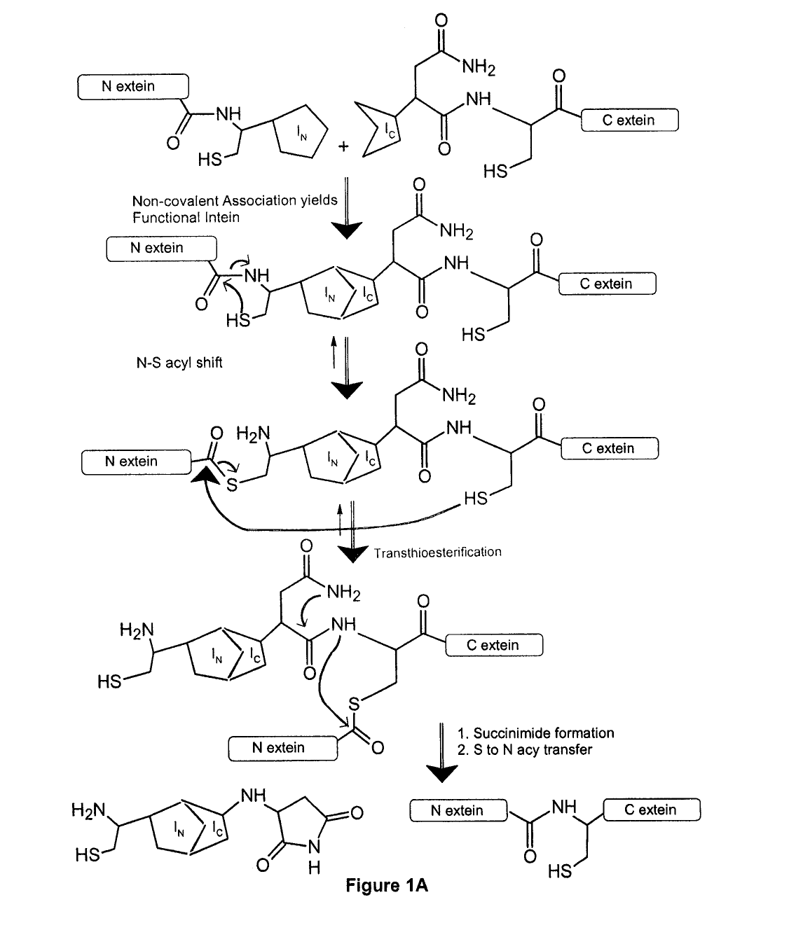

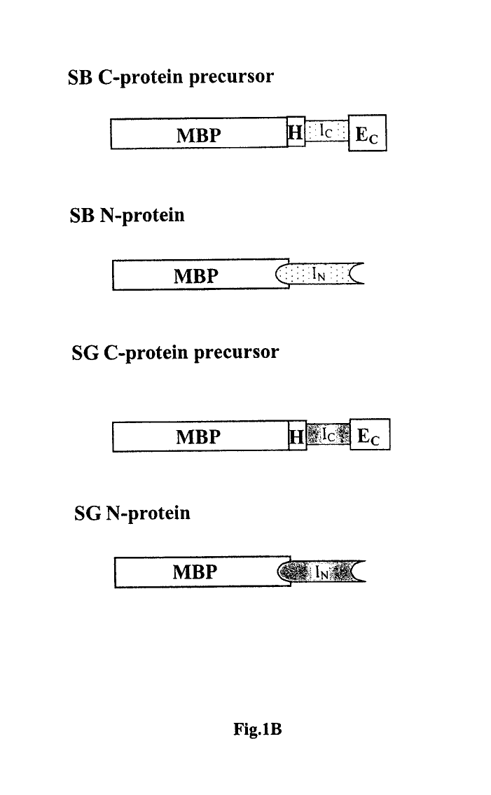

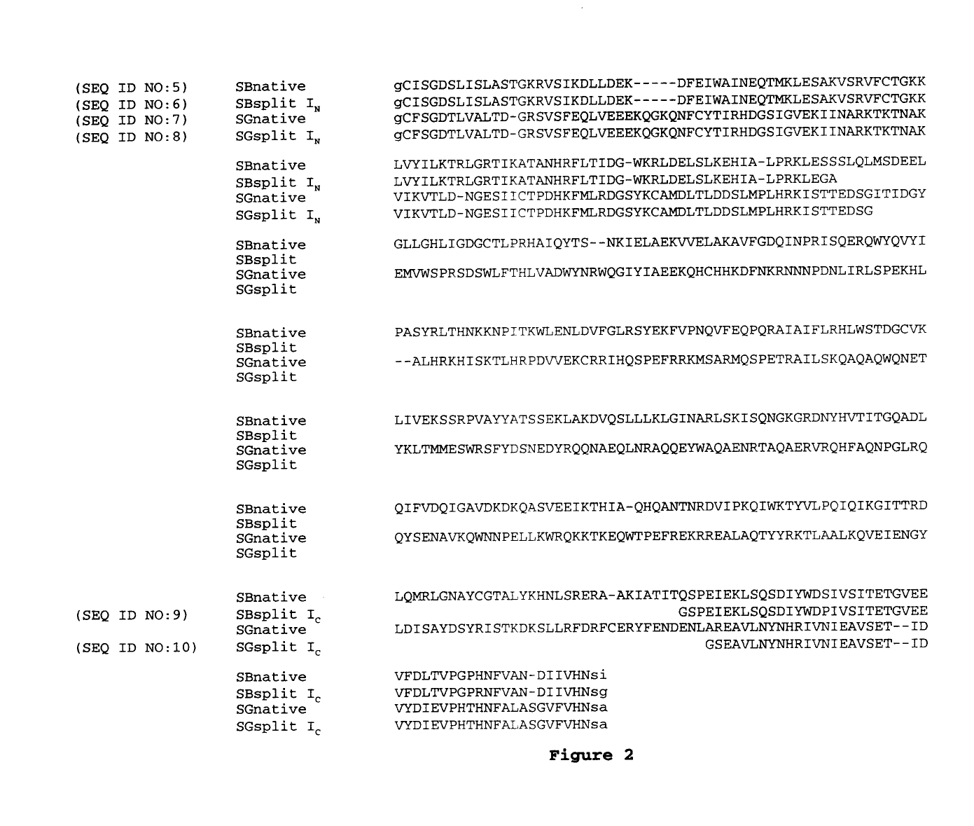

Protein trans-splicing (PTS) utilizes protein trans-splicing elements known as “split inteins”. The principle of PTS is illustrated in FIG. 1A: two components of a split intein, termed the N-intein (I_{N}) and the C-intein (I_{C}), associate and fold to form a functional intein, which can then undergo a pseudo-intramolecular protein splicing reaction, wherein the flanking polypeptides, termed the N-extein (E_{N}) and C-extein (E_{C}) are ligated together and the intein excises itself (see FIG. 1A). For a recent review on protein splicing and PTS, see Vasant Muralidharan and Tom W. Muir (Nature Methods. (2006) Vol. 3 No. 6 pp. 429-438).

In the present method, one or more water-soluble polymer molecules can be attached to the carrier molecule either before or after the trans-splicing reaction, provided that the attached water soluble polymer molecules do not prevent subsequent protein trans-splicing activity.

However, in many cases, it is desirable and advantageous to attach the polymer to the carrier molecule before the trans-splicing reaction, for example so as to attach the polymer specifically to the carrier molecule and avoid unwanted attachment to the target polypeptide, or so as to protect the target polypeptide from the chemical conditions used in the attachment process.

Another advantage of the present invention is that it permits one to link the carrier molecule/polymer conjugate to either the carboxy-terminal or amino-terminal of the target polypeptide, by designing appropriate trans-splicing partners.

As used herein, both “protein” and “polypeptide” mean any chain of amino acids, regardless of length or post-translational modification (e.g. glycosylation or phosphorylation, etc.), and include natural proteins, synthetic or recombinant polypeptides and peptides, as well as a recombinant molecule consisting of a hybrid comprising two polypeptide segments that are encoded by all or part of a hybrid nucleotide sequence.

Herein, the term “PEGylation” describes the conjugation of a water-soluble poilymer molecule (such as PEG, MPEG, and the like) to a polypeptide by way of a covalent bond.

An “intein” is a segment of a polypeptide that is able to excise itself and join the remaining portions (called “exteins”) of the polypeptide with a peptide bond. Thus, an intein is a protein splicing element, i.e. an amino acid sequence that has polypeptide-splicing enzymatic activity. As is known in the art, intein functionality can be provided by a single polypeptide that can undergo an intramolecular protein splicing reaction; or intein functionality can be “split” between two polypeptide components that can associate to form a functional intein that undergoes an intermolecular protein trans-splicing (PTS) reaction to join two extein segments. More particularly, such “split inteins” comprise an N-terminal component and a C-terminal component, which are also referred to herein as an “N-intein (I_{N})” or a “C-intein (I_{C})”, respectively. Thus, in the case of a prtein trans-splicing (PTS) reaction utilizing split intein, there is a pair of polypeptides, referred to herein as an “N-protein” and a “C-protein”, each of which comprises an extein segment and a split intein component. The N-protein comprises an amino-terminal extein segment (an N-extein or E_{N}) fused at its carboxy-terminal residue to an N-intein (I_{N}) split intein component, and the “C-protein” comprises a C-intein (I_{C}) split intein component followed by a carboxy-terminal extein segment (a C-extein or E_{C}) (as illustrated in FIG. 1A). In accordance with the method of the present invention, one of the exteins of such a pair of N- and C-proteins will comprise a target polypeptide and the other will comprise a carrier molecule for attaching a water-soluble polymer.

Herein, the term “splicing residue” refers to the C-terminal residue of the N-extein (E_{N}) segment of the N-protein and the N-terminal residue of the C-extein (E_{C}) segment of the C-protein. The splicing residues are directly involved in the molecular rearrangement that ligates the exteins together and excises the N- and C-split intein components. Note that the splicing residues are included in the N-extein/C-extein ligation product and are linked to each other by the newly formed amide bond.

Herein, the term as “trans-splicing partners” refers to an N-protein and C-protein pair having respectively N-intein (I_{N}) and C-intein (I_{C}) components that are capable of interacting to provide the intein function, wherein one of N- or C-proteins has an extein segment comprising the target polypeptide and the other comprises an extein comprising a carrier molecule for attaching a water-soluble polymer. The term “trans-splicing partner” refers to one of such pair of polypeptides. The trans-splicing partners may be referred to more specifically herein as the “target polypeptide trans-splicing partner” and the “carrier molecule trans-splicing partner”.

### A. Preparation of Trans-Splicing Partners

The method of the present invention begins with the preparation of suitable trans-splicing partners. The trans-splicing partners can be prepared using routine molecular biology techniques (e.g. via prokaryotic or eukaryotic host expression of exogenous synthetic or recombinant DNA sequences) or using chemical synthesis. DNA molecules that encode the trans-splicing partners, and expression vectors comprising them, can be prepared using conventional methods. In general, any expression vector and supporting host can be used to express the trans-splicing partners. It is within the ability of persons skilled in the art of protein expression and availed of the teaching herein to design appropriate DNA molecules encoding appropriate trans-splicing partners for practicing the invention, and choose an appropriate expression system for expressing them.

In some embodiments, the trans-splicing partner is initially expressed in the form of a precursor polypeptide that comprises additional elements that are typically removed (cleaved) prior to the protein trans-splicing reaction. For example, the precursor polypeptide may comprise an affinity tag (such as a histidine tag) to assist in purification or a supporting protein (like maltose binding protein) that can be removed to produce the desired trans-splicing partner. Such affinity tags can also be advantageously incorporated into reagents by appending to the free end of a split intein component (i.e. the end not attached to the extein) attached and either directly attached or attached with a spacer polypeptide sequence, provided that such attachments allow the splicing reaction to proceed without the requirement of cleavage of the affinity tag prior to splicing. Since the splicing reaction does, in fact, remove all non-extein fragments from the target protein, any carrier molecule reagent or target protein with such an affinity tag appended to the intein portion of its structure will have the tag removed by the splicing reaction. This will allow any excess or unreacted intein-containing byproducts to be removed by their affinity tags. Only spliced product would lack the affinity tag and have the water-soluble polymer-conjugated extein attached. Thus, examples of suitable C-protein trans-splicing partners which comprise carrier molecules for attaching a water-soluble polymer include but are not limited to a polypeptide comprising an amino acid sequence as set forth in residues 388 to 453 of SEQ ID NO:1, 398 to 453 of SEQ ID NO:1, 398 to 449 of SEQ ID NO:3, or 388 to 449 of SEQ ID NO:3, or a conjugate thereof that is attached to at least one water-soluble polymer. Examples of suitable N-protein trans-splicing partners which comprise a target protein include but are not limited to a polypeptide comprising an amino acid sequence as set forth in SEQ ID NO:2 or SEQ ID NO:4.

### B. Split Inteins:

In general, the trans-splicing partners can be designed using any split intein, including any naturally-occurring or artificially-split split intein. Several naturally-occurring split inteins are known, for example: the split intein of the DnaE gene of Synechocystis sp. PCC6803 (see Wu H, Hu Z, Liu X Q. “Protein trans-splicing by a split intein encoded in a split DnaE gene of Synechocystis sp. PCC6803.” Proc Natl Acad Sci USA. (1998) 95(16):9226-31; and Evans T C Jr, Martin D, Kolly R, Panne D, Sun L, Ghosh I, Chen L, Benner J, Liu X Q, Xu M Q. “Protein trans-splicing and cyclization by a naturally split intein from the dnaE gene of Synechocystis species PCC6803. J Biol Chem. (2000) 275(13):9091-4 and of the DnaE gene from Nostoc punctiforme (see Iwai H, Zuger S, Jin J, Tam P H. “Highly efficient protein trans-splicing by a naturally split DnaE intein from Nostoc punctiforme.” FEBS Lett. (2006) 580(7):1853-8). Non-split inteins have been artificially split in the laboratory to create new split inteins, for example: the artificially split Ssp DnaB intein (see Wu H, Xu M Q, Liu X Q. “Protein trans-splicing and functional mini-inteins of a cyanobacterial dnaB intein.” Biochim Biophys Acta. (1998)1387 422-32) and split Sce VMA intein (see Brenzel S, Kurpiers T, Mootz H D. “Engineering artificially split inteins for applications in protein chemistry: biochemical characterization of the split Ssp DnaB intein and comparison to the split Sce VMA intein.” Biochemistry. (2006)45(6):1571-8); and an artificially split fungal mini-intein (see Elleuche S, Poggeler S. “Trans-splicing of an artificially split fungal mini-intein.” Biochem Biophys Res Commun. (2007) 355(3):830-4). There are also intein databases available that catalogue known inteins (see for example the online-database available at: http://bioinformatics.weizmann.ac.il/˜pietro/inteins/Inteins table.html). Naturally-occurring non-split inteins may have endonuclease or other enzymatic activities that can typically be removed when designing an artificially-split split intein. Such mini-inteins or minimized split inteins are well known in the art and are typically less than 200 amino acid residues long (see Wu H, Xu M Q, Liu X Q. “Protein trans-splicing and functional mini-inteins of a cyanobacterial dnaB intein.” Biochim Biophys Acta. (1998)1387 422-32). Suitable split inteins may have other purification enabling polypeptide elements added to their structure, provided that such elements do not inhibit the splicing of the split intein or are added in a manner that allows them to be removed prior to splicing. Protein splicing has been reported using proteins that comprise bacterial intein-like (BIL) domains (see Amitai G, Belenkiy O, Dassa B, Shainskaya A, Pietrokovski S. “Distribution and function of new bacterial intein-like protein domains.” Mol Microbiol. (2003) 47 61-73) and hedgehog (Hog) auto-processing domains (the latter is combined with inteins when referred to as the Hog/intein superfamily or HINT family (see Dassa B, Haviv H, Amitai G, Pietrokovski S. “Protein splicing and auto-cleavage of bacterial intein-like domains lacking a C′-flanking nucleophilic residue” J Biol Chem. (2004) 279 32001-7); and domains such as these may also be used to prepare artificially-split inteins. In particular, non-splicing members of such families may be modified by molecular biology methodologies to introduce or restore splicing activity in such related species. Recent studies demonstrate that splicing can be observed when a N-terminal split intein component is allowed to react with a C-terminal split intein component not found in nature to be its “partner”; for example, splicing has been observed utilizing partners that have as little as 30 to 50% homology with the “natural” splicing partner (see Dassa B, Amitai G, Caspi J, Schueler-Furman O, Pietrokovski S. “Trans protein splicing of cyanobacterial split inteins in endogenous and exogenous combinations.” Biochemistry. (2007) 46(1):322-30). Other such mixtures of disparate split intein partners have been shown to be unreactive one with another (see Brenzel S, Kurpiers T, Mootz H D. “Engineering artificially split inteins for applications in protein chemistry: biochemical characterization of the split Ssp DnaB intein and comparison to the split Sce VMA intein.” Biochemistry. 2006 45(6):1571-8). However, it is within the ability of a person skilled in the relevant art to determine whether a particular pair of polypeptides is able to associate with each other to provide a functional intein, using routine methods and without the exercise of inventive skill.

Known inteins (including split inteins) have a relatively diverse makeup. This is a well studied area and has been reviewed and the critical and conserved aspects of inteins have been described (see Saleh L, Perler F B. “Protein splicing in cis and in trans.” Chem Rec. (2006) 6 183-93). One of the most conserved requirements for splicing is the presence of a serine, cysteine or threonine residue as the splicing residue present at the N-terminal end of the C-extein, while a wide variety of amino acids are known to be functional in splicing at the C-terminal end of the N-extein. In fact, the mutation of a C-extein to one that lacks this N-terminal cysteine, serine or threonine has been used to render a splicing intein inactive. This is the basis for the non-splicing forms marketed for protein purification applications requiring fission, not splicing (see Mathys S, Evans T C, Chute I C, Wu H, Chong S, Benner J, Liu X-Q, Xu M-Q. “Characterization of a self-splicing mini-intein and its conversion into autocatalytic N- and C-terminal cleavage elements: facile production of protein building blocks for protein ligation.” Gene. (1999) 231 1-13). Other highly conserved residues may be present in functional split inteins useful in the present method. Specifically, most but not all known inteins comprise a cysteine, serine or threonine at the N-terminus of the N-intein (i.e. at the position adjacent to the splicing residue located at the C-terminus of the N-extein). However, this N-terminal residue of the N-intein can be varied; for example, a listing of seven inteins having an alanine at this site as well as a discussion of the mechanism of splicing in the presence of this variation has been published (see Southworth M W, Adam E, Panne D, Byer R, Kautz R, Perler F B. “Control of protein splicing by intein fragment reassembly.” EMBO J. (1998) 17 918-26). Notably, then, the N-terminal residue of the N-intein and the splicing residue of the C-extein can be the same species or different species. Further, the C-terminal end of the C-intein of a split intein may comprise an asparagine residue that is highly conserved. The penultimate residue at the C-terminal end of the C-intein of a split intein splicing pair is most often histidine; while highly conserved, there are reported inteins with phenylalanine, glycine, alanine, serine, lysine present in the penultimate position of the C-terminus of the C-intein and glutamine and aspartic acid residues have replaced the ultimate asparagines in reported inteins (see Chen L, Benner J, Perler F B. “Protein splicing in the absence of an intein penultimate histidine.” J Biol Chem. (2000) 275(27):20431-5; and Amitai G, Dassa B, Pietrokovski S. “Protein splicing of inteins with atypical glutamine and aspartate C-terminal residues.” J Biol Chem. (2004) 279 3121-31). One may see these highly conserved residues in the sequences of FIG. 1 in U.S. Pat. No. 5,834,247 to Combs et. al. In many cases, His-Asn will be the penultimate and ultimate N-terminal residues of the C-intein, as these residues are highly conserved across inteins.

In as much as some split inteins have functional components small enough to be produced synthetically instead of by protein expression in vivo, it will be apparent to one skilled in the art that the extein of a synthetically produced trans-splicing partner may have a greater variety of possible types of sites for polymer conjugation in the above structures than the natural or unnatural amino acids. The split intein component of such splicing partners can also be subject to modification from the naturally occurring and known protein splicing elements by such methods as directed evolution and selective or unselective mutations as are commonly practiced in optimizing or modifying the behavior of protein elements. Amitai describes the production of several mutants of natural inteins, some with improved and some with hindered reactivity (see Amitai G, Dassa B, Pietrokovski S. “Protein splicing of inteins with atypical glutamine and aspartate C-terminal residues.” J Biol Chem. (2004) 279 3121-31). Iwai et al. give an even more comprehensive example of such mutation or protein engineering of a split intein (see Iwai H, Zuger S, Jin J, Tam P H. “Highly efficient protein trans-splicing by a naturally split DnaE intein from Nostoc punctiforme.” FEBS Lett. (2006) 580(7):1853-8). The published demonstration that the N-terminal intein component of one split intein can be combined with the C-terminal intein component of a different split intein and still yield spliced products from their respective extein fragments and the published report that a Ssp DnaB mini-intein could be split in several different sites and could be split into a three piece split intein, each piece required for splicing, demonstrates that a variety of constructs can be functional in the present invention, with the key attribute being the design of one of the exteins to allow specific conjugation with a water soluble polymer either before or after splicing (see Iwai H, Zuger S, Jin J, Tam P H. “Highly efficient protein trans-splicing by a naturally split DnaE intein from Nostoc punctiforme.” FEBS Lett. (2006) 580(7):1853-8; Dassa B, Amitai G, Caspi J, Schueler-Furman O, Pietrokovski S. “Trans protein splicing of cyanobacterial split inteins in endogenous and exogenous combinations.” Biochemistry. (2007) 46(1):322-30; and Sun W, Yang J, Liu X Q. “Synthetic two-piece and three-piece split inteins for protein trans-splicing.” J Biol Chem. (2004) 279 35281-6).

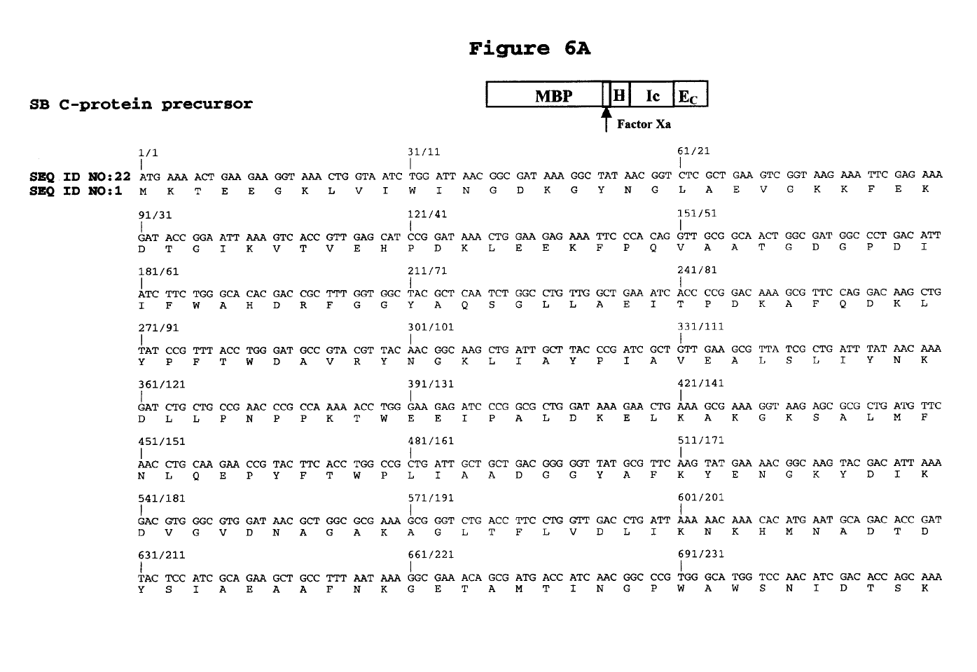

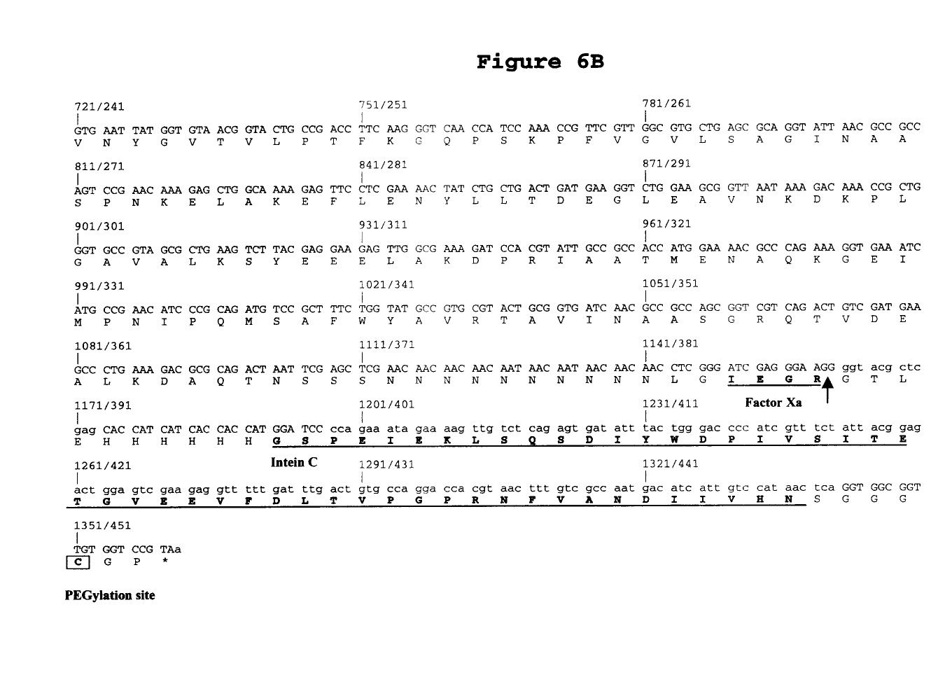

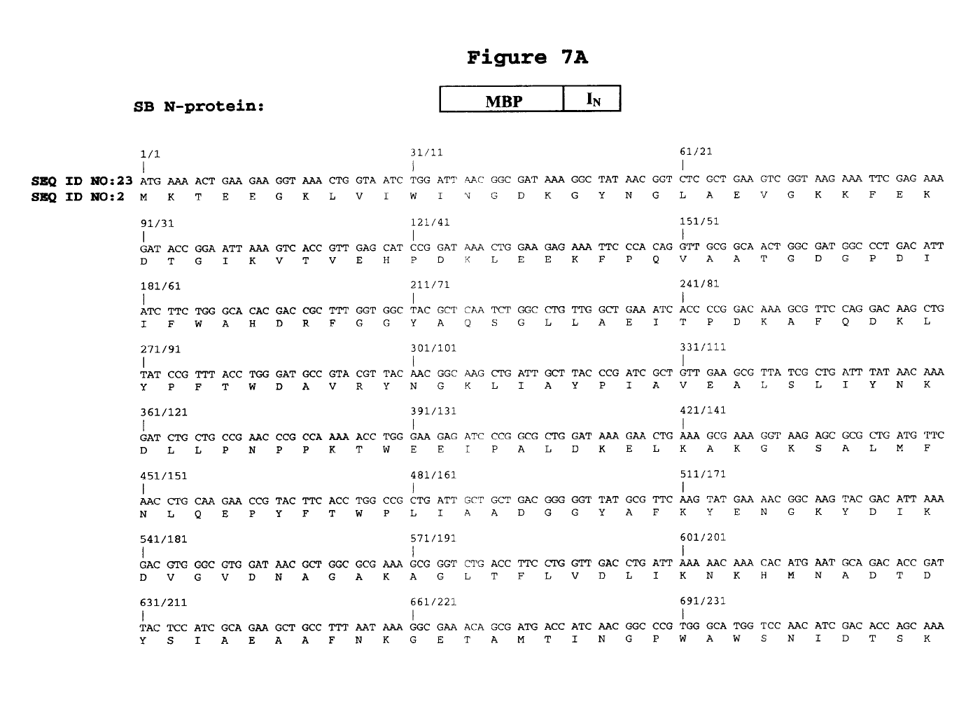

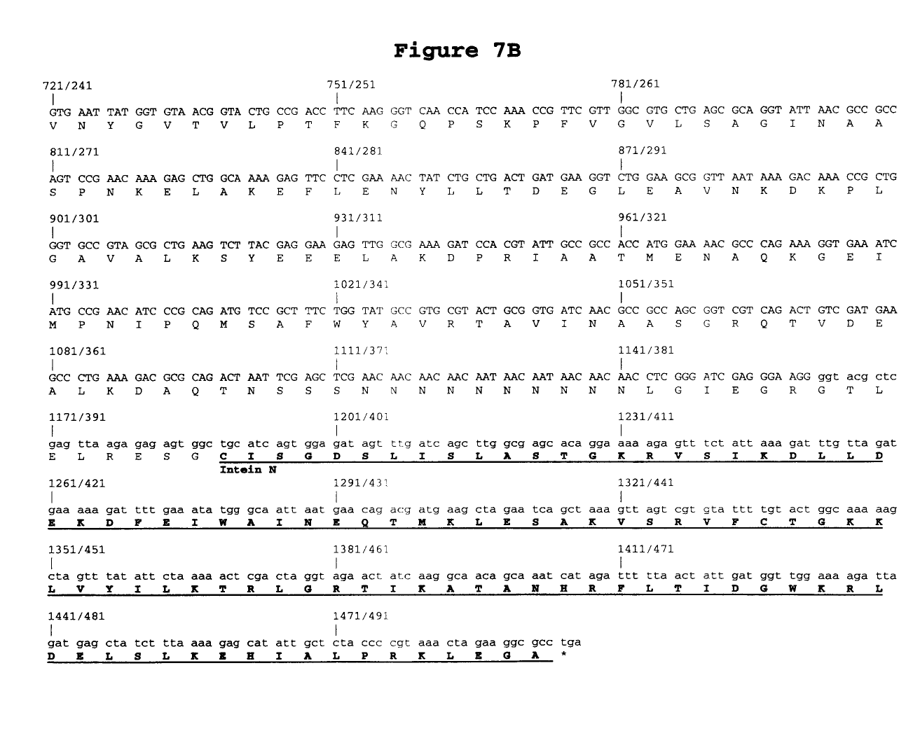

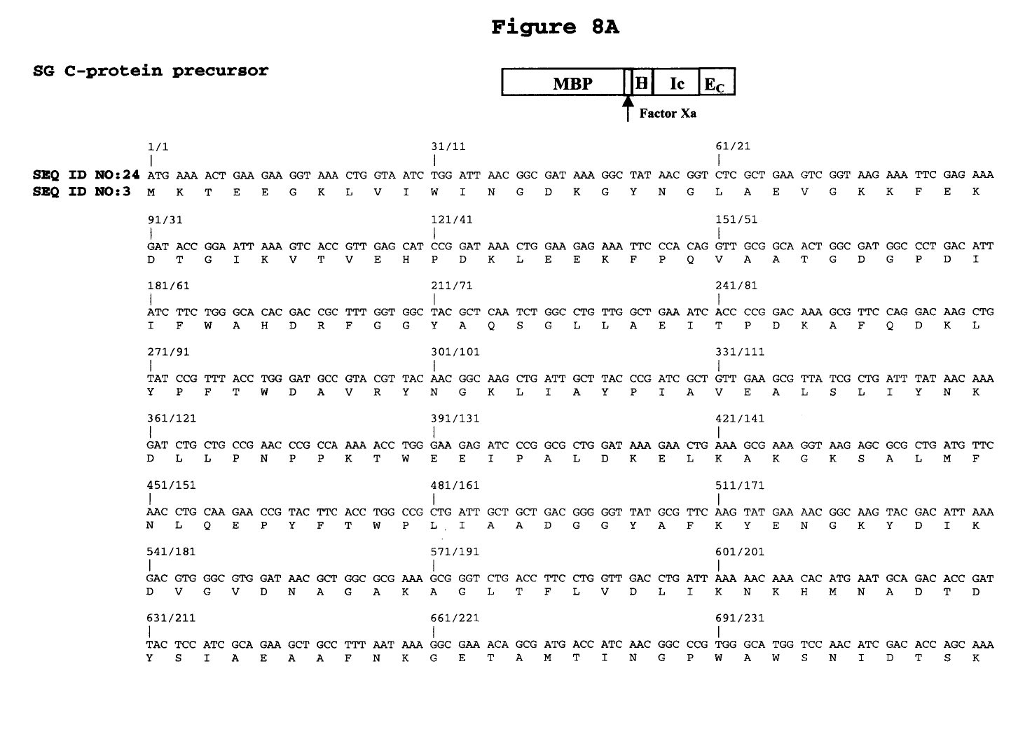

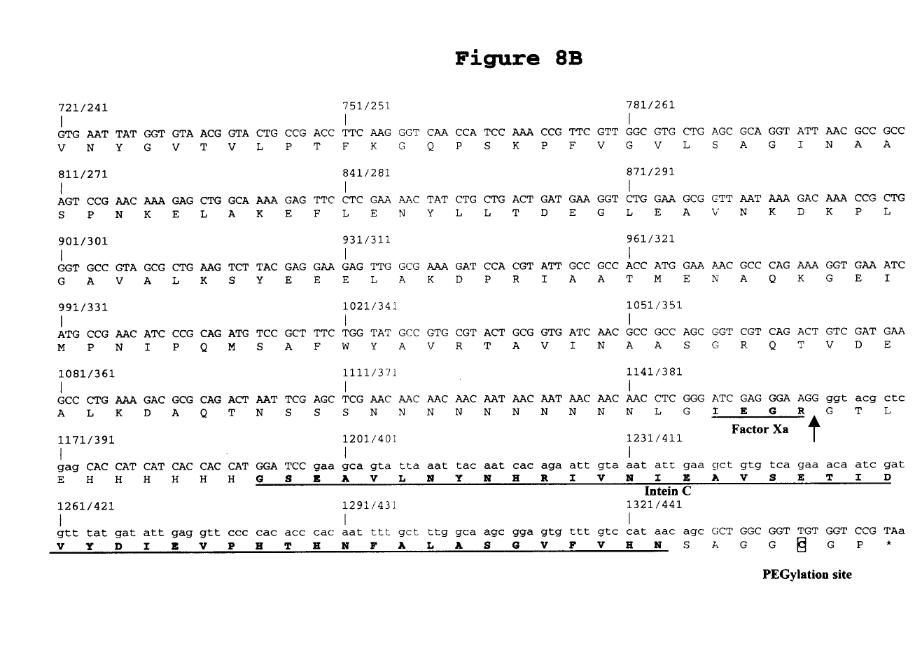

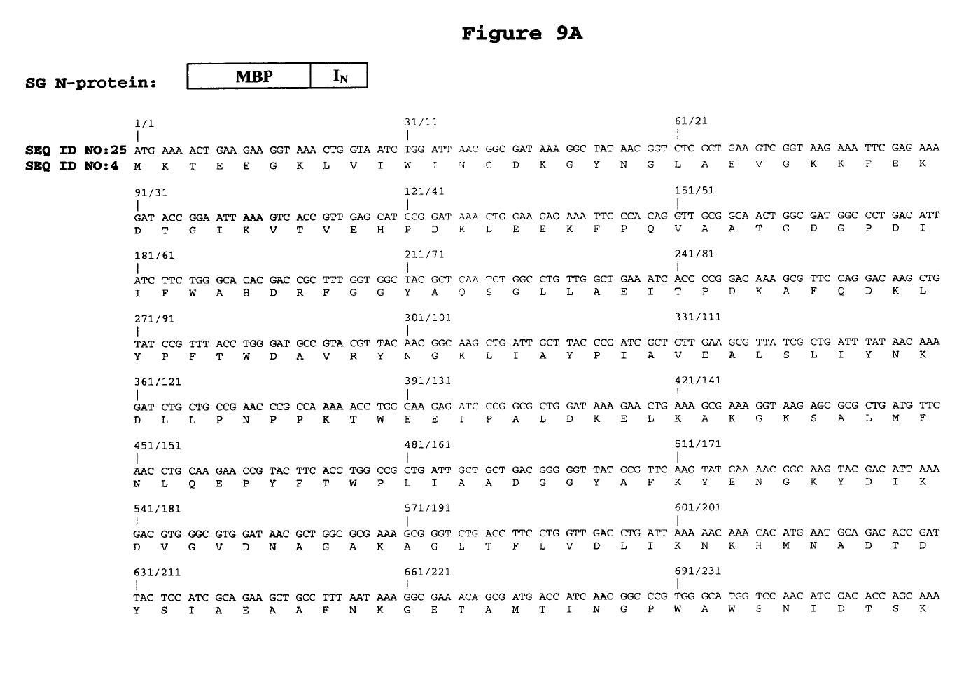

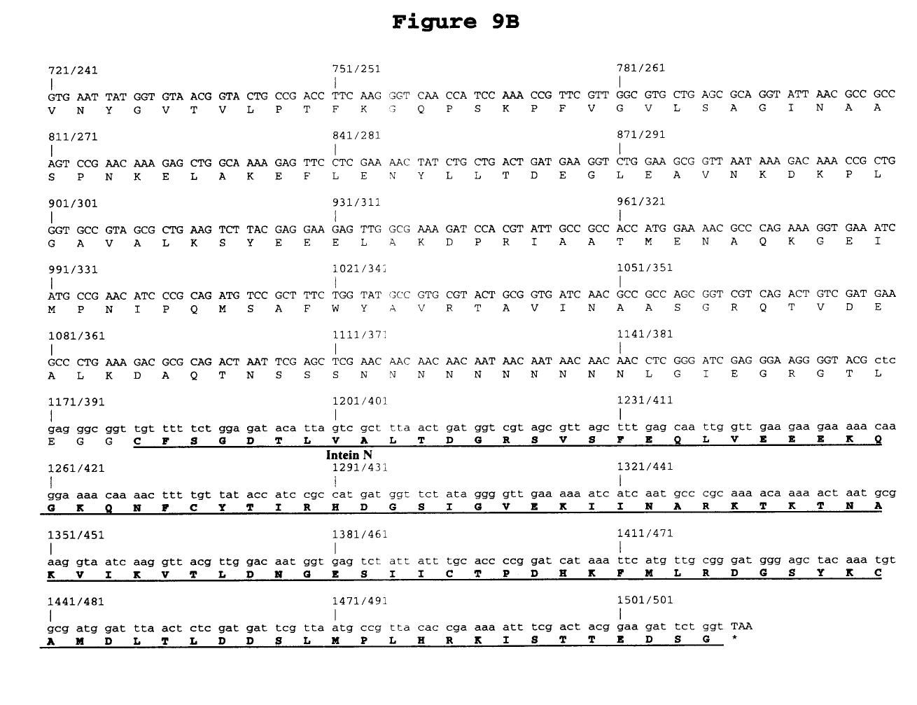

The sequences specifically disclosed in this invention include many of the highly conserved features mentioned above. SB C-protein (residues 388 to 453 of SEQ ID NO:1) and SG C-protein (residues 388 to 449 of SEQ ID NO:3) both have a serine residue that is the splicing residue at the N-terminus of the C-extein, and both exhibit the penultimate histidine and ultimate asparagines at the C terminus of the C-intein segment (see FIGS. 6B and 8B). Their trans-splicing partners SB N-protein (SEQ ID NO:2) and SG N-protein (SEQ ID NO:4) both exhibit an N-terminal cysteine on the N-intein segment, adjacent to the splicing residue of the extein (see FIGS. 7B and 9B). However, while the most common features of the currently known split intein splicing pairs will often be present in any implementation of the invention, they can be varied (as discussed above) provided that the pair of splicing partners remains capable of trans-splicing in vitro.

Thus, the method of the present invention can be practiced using naturally-occurring split inteins, artificially-split split inteins, or functional variants thereof wherein the amino acid sequence of either of both of the I_{N} and I_{C} components of the split intein has at least 30%, at least 40%, at least 50%, at least 60%, at least 70%, at least 75%, at least 80%, at least 85%, at least 90%, at least 95% identity to the native sequence of the respective I_{N} and I_{C} components of the naturally-occurring split intein or artificially-split split intein.

Examples of suitable split inteins for practicing the method of the invention include but are not limited to:

(a) a split intein comprising SBsplit I_{N} (residues 2 to 103 of SEQ ID NO:6) and SBsplit I_{C} (residues 1 to 49 of SEQ ID NO:9), or functional variants thereof;

(b) a split intein comprising SGsplit I_{N} (residues 2 to 112 of SEQ ID NO:8) and SGsplit I_{C} (residues 1 to 45 of SEQ ID NO:10), or functional variants thereof;

(c) a split intein from the split DnaE gene of Synechocystis sp. PCC6803 (see Wu H, Hu Z, Liu X Q. “Protein trans-splicing by a split intein encoded in a split DnaE gene of Synechocystis sp. PCC6803.” Proc Natl Acad Sci USA. (1998) 95(16):9226-31), or a functional variant thereof;

(d) an artificially split Ssp DnaB intein (see Wu H, Xu M Q, Liu X Q. “Protein trans-splicing and functional mini-inteins of a cyanobacterial dnaB intein.” Biochim Biophys Acta. (1998)1387 422-32; and Brenzel S, Kurpiers T, Mootz H D.“Engineering artificially split inteins for applications in protein chemistry: biochemical characterization of the split Ssp DnaB intein and comparison to the split Sce VMA intein.” Biochemistry. (2006)45(6):1571-8, or a functional variant thereof;

(e) an artificially split Sce VMA intein (see Brenzel S, Kurpiers T, Mootz H D. “Engineering artificially split inteins for applications in protein chemistry: biochemical characterization of the split Ssp DnaB intein and comparison to the split Sce VMA intein.” Biochemistry. (2006)45(6):1571-8), or a functional variant thereof;

(f) an artificially split fungal mini-intein (see Elleuche S, Poggeler S. “Trans-splicing of an artificially split fungal mini-intein.” Biochem Biophys Res Commun. (2007) 355(3):830-4), or a functional variant thereof; and

(g) Npu DanE split intein (see Iwai H, Zuger S, Jin J, Tam P H. “Highly efficient protein trans-splicing by a naturally split DnaE intein from Nostoc punctiforme.” FEBS Lett. (2006) 580(7):1853-8), or a functional variant thereof.

Suitable split inteins also include but are not limited to split inteins derived from the M. tuberculosis RecA intein and its several reported modified forms (see Lew B M, Mills K V, Paulus H. “Characteristics of protein splicing in trans mediated by a semisynthetic split intein. “Biopolymers. (1999) 51 355-62), the DnaE_{C} split intein (see Wu H, Xu M Q, Liu X Q. “Protein trans-splicing and functional mini-inteins of a cyanobacterial dnaB intein.” Biochim Biophys Acta. (1998)1387 422-32) and the more recently described NpuDnaE split intein and modifications or variants of it (see Iwai H, Zuger S, Jin J, Tam P H. “Highly efficient protein trans-splicing by a naturally split DnaE intein from Nostoc punctiforme.” FEBS Lett. (2006) 580(7):1853-8).

### C. The Target Polypeptide

In general, the method of the invention can be practiced using any target polypeptide that can be produced in active form using chemical synthesis or heterologous protein expression techniques. In some cases, it may be necessary to re-fold the target polypeptide either before or after ligating it to the carrier molecule, in order to provide the active form.

As discussed above, the extein of one of the trans-splicing partners will comprise a target molecule, and the extein of the other trans-splicing partner will comprise a “carrier molecule” for attaching at least one water-soluble polymer molecule. In some embodiments, the target polypeptide will be a polypeptide of interest (i.e. having a bioactivity of interest) and the carrier molecule will be an exogenous single amino acid or polypeptide. However, in other embodiments, the target polypeptide and the carrier molecule will both be derived from the sequence of the polypeptide of interest.

As discussed above, the sequence of the C-extein must include an N-terminal amino acid that can serve as a splicing residue, and the N-extein must contain a C-terminal amino acid that can serve as a splicing residue. The required splicing residue can be provided by the native sequence of the target polypeptide or by adding an appropriate N-terminal or C-terminal residue to the sequence of the target polypeptide.

Also, it is known that the amino acid sequences of the N- or C-exteins immediately adjacent to the split intein component can affect splicing efficiency, and the sequence of the N-extein and/or C-extein can be chosen or designed or varied with this in mind. For example, the effect of the penultimate extein residue of the C-extein has been studied for the DnaEc split intein (see Iwai H, Zuger S, Jin J, Tam P H. “Highly efficient protein trans-splicing by a naturally split DnaE intein from Nostoc punctiforme.” FEBS Lett. (2006) 580(7):1853-8) and demonstrates the residue adjacent to the splicing site can influence splicing efficiency. This study found that having tyrosine or phenylalanine or tryptophan as the penultimate extein residue of the C-extein increased the coupling efficiency in the systems studied, and consequently C-exteins of the present invention may have have tyrosine, phenylalanine or tryptophan as the penultimate extein residue . In the case of the N-extein, certain carboxy-terminal splicing residues have been shown to be more efficient in related native protein ligation studies (see Hackeng T M, Griffin J H, Dawson P E. “Protein synthesis by native chemical ligation: expanded scope by using straightforward methodology.” Proc Natl Acad Sci USA. (1999) 96 10068-73) and may be expected to yield higher splicing efficiency in intein mediated ligation as well. In such studies, model systems having glycine, cysteine and histidine were found to slightly outperform systems with phenylalanine, alanine, tryptophan, tyrosine, and methionine, with slower splicing and lower efficiency being observed with the other amino acids. Splicing was reported for model compounds having all natural amino acids as the carboxy-terminal residue (see Hackeng T M, Griffin J H, Dawson P E. “Protein synthesis by native chemical ligation: expanded scope by using straightforward methodology.” Proc Natl Acad Sci USA. (1999) 96 10068-73). In exemplary embodiments, the carboxy-terminal splicing residue of the N-extein is glycine, but other inteins show that a variety of residues (including serine and threonine) can be functional as the splicing residue of the N-extein.

### D. The Carrier Molecule

As discussed above, the extein of one of the trans-splicing partners will comprise a target polypeptide, and the extein of the other trans-splicing partner will comprise a “carrier molecule”.

The “carrier molecule” is an amino acid or polypeptide that contains at least one functional group (attachment site) that is suitable for covalently attaching a water-soluble polymer molecule. In many cases, the carrier molecule will have a single attachment site for attaching a water-soluble polymer molecule. However, the carrier molecule can have two, three or more attachment sites for attaching two, three or more water-soluble polymer molecules. In general, any polypeptide that has one or more suitable attachment sites can be used in the present invention. However, in many cases, the carrier molecule will be a small polypeptide having between 2 and 30 amino acids, and preferably between 2 and 20 amino acids (e.g. having 2, 3, 4, 5, 6, 7, 8, 9, 10, 11, 12, 13, 14, 15, 16, 17, 18, 19, or 20 amino acid residues).

In general, the invention can be practiced using carrier molecules having any of a variety of functional groups for use as attachment sites. In many cases, the attachment site will be provided by amino acid that has a functional group suitable for use as an attachment site. Examples of suitable amino acids for this purpose include the following naturally occurring amino acids: lysine, cysteine, histidine, arginine, aspartic acid, glutamic acid, serine, threonine, tyrosine. Unnatural amino acids such as para-acetyl-phenylalanine (pAcF) and other ketone containing amino acids, homocysteine or selenocysteine can also serve as attachment sites. The N-terminal amino group and the C-terminal carboxylic acid can also be used as attachment sites. Water-soluble polymer molecules can be attached to attachment sites using any suitable method, and a variety of such methods are known (as further discussed below).

If a water-soluble polymer molecule is to be attached to the carrier molecule prior to the PTS reaction, then the trans-splicing partner will generally be designed so that the amino acids serving as splicing residue and target sites for attaching polymer are different, so that the water-soluble polymer can be attached to the target residue (and not the splicing residue) in a substantially selective manner. For example, the carrier molecule can have one or more cysteine residues to serve as target sites for attaching water-soluble polymers and a serine (not a cysteine) to serve as a splicing residue. Thus, the water-soluble polymer can be attached to the target cysteine residue (and not the splicing residue) in a substantially selective manner, prior to the PTS reaction.

However, depending on the sequence of the target polypeptide, the carrier molecule can be chosen so that it presents one or more unique residues (such as an unpaired Cys) as target sites to allow attachment of water-soluble polymer molecules after the PTS reaction that is substantially specific.

The carrier molecule may also comprise amino acids (such as Ala and Gly) that function as spacing elements, to provide space between the splice residue and the attachment site or sites. The carrier molecule may also comprise one or more residues located beyond the attachment site (such as a terminal proline residue) that may serve to inhibit proteolysis.

In some embodiments, the carrier molecule is exogenous to the polypeptide of interest. For example, the carrier molecule can be a short artificial sequence (for example as set forth in SEQ ID NO:16) or one or more repeats thereof.

In other embodiments, the carrier molecule can be derived from the sequence of a polypeptide of interest, which is split to provide both of the carrier molecule and the target polypeptide (or the exteins comprising them). Using this approach, it is possible to minimize the changes made to the sequence of the polypeptide of interest, by adding as little as zero, one or two amino acids to the polypeptide of interest, and yet achieve the desired modification of adding a water-soluble polymer molecule to the polypeptide of interest.

The carrier molecule can optionally be fused or linked to other polypeptide elements (e.g. purification tags or other polypeptides), that together make up the extein segment.

A carrier molecule sequence may be shortened or extended to achieve high efficiency of the coupling and to optimize the biological activity of the spliced product.

As discussed above, the sequences of the C-extein and N-extein must each include an amino acid that can serve as a splicing residue. The required splicing residue may be provided by the sequence of the carrier molecule or added thereto.

Also as discussed above, it is known that the amino acid sequences of the N- or C-exteins immediately adjacent to the split intein can affect splicing efficiency, and the sequence of the N- and/or C-extein may be chosen or designed or varied with this in mind.

Thus, the method of the invention can be practiced for example using carrier molecule comprising, but not limited to, a polypeptide having the following general structure (SEQ ID NO:15):

| | |
|---|---|
| Xaa Xaa Xaa Xaa Xaa Xaa Xaa Xaa Xaa Xaa Xaa Xaa Xaa Xaa Xaa | 15 |
| Xaa Xaa Xaa Xaa Xaa Xaa Xaa Xaa Xaa Xaa Xaa Xaa Xaa Xaa Xaa | 30 |
| Xaa Xaa Xaa Xaa Xaa Xaa Xaa Xaa Xaa Xaa Xaa Xaa Xaa Xaa Xaa | 45 |
| Xaa Xaa Xaa Xaa Xaa Xaa Xaa Xaa Xaa Xaa Xaa Xaa Xaa Xaa Xaa | 60 |
| Xaa Xaa Xaa Xaa Xaa Xaa Xaa Xaa Xaa Xaa Xaa Xaa Xaa Xaa Xaa | 75 |
| Xaa Xaa Xaa Xaa Xaa Xaa Xaa, | |

wherein:

Xaa at positions 1 to 39 can be any amino acid (e.g. Ala or Gly) or absent;

Xaa at position 40 is an amino acid suitable for conjugation to a water-soluble polymer, such as Cys, Lys, His, Arg, Asp, Glu, Ser, Thr, Tyr, or a ketone-containing unnatural amino acid such as para-acetyl-phenylalanine (pAcF), homocysteine, or selenocysteine;

Xaa at positions 41 to 82 can be any amino acid (e.g. Ala or Gly) or absent.

Xaa at positions 1 to 39 and 41 to 82 of SEQ ID NO:15 can be any amino acid but are generally chosen so that they provide spacing between the sites for attaching water-soluble polymers and other functional elements of the extein or other functionality (as discussed below). Mention is made of Ala and Gly as suitable amino acids for use as spacing residues.

In embodiments (for example when the N-protein comprises the carrier molecule), one or more of Xaa residues at positions 1 to 39 (e.g. the amino-terminal residue) of SEQ ID NO:15 is chosen to be resistant to proteases found in human serum, plasma or blood.

In embodiments, at least one Xaa at positions 1 to 39 or 41 to 80 of SEQ ID NO:15 is an amino acid suitable for conjugation to a water-soluble polymer, as described above, thereby providing at least one additional site for attaching a water-soluble molecule to the carrier molecule.

Thus, in embodiments, the N-extein or C-extein can comprise a carrier molecule having the following sequence (SEQ ID NO:18), or one or more repeats thereof:

Xaa Xaa Xaa Xaa Xaa Xaa

wherein:

Xaa at positions 1 and 2 can be any amino acid (e.g. Ala or Gly);

Xaa at position 3 is an amino acid suitable for conjugation to a water-soluble polymer, such as Cys, Lys, His, Arg, Asp, Glu, Ser, Thr, Tyr, or a ketone-containing unnatural amino acid such as para-acetyl-phenylalanine (pAcF), homocysteine, or selenocysteine; and

Xaa at positions 4 to 6 can be any amino acid (e.g. Ala or Gly).

In embodiments, when Xaa at position 1 of SEQ ID NO:18 is the N-terminal residue of the N-extein, it can be a proteolysis-inhibiting amino acid such as Pro. In embodiments, when Xaa at position 6 is the C-terminal residue of the C-extein, it can be a proteolysis-inhibiting amino acid such as Pro.

Examples of N-exteins comprising carrier molecules therefore include but are not limited to: PGCGGG (SEQ ID NO:16) and PGCGGA (SEQ ID NO:17).

In embodiments, the C-extein of the C-protein can be a carrier molecule of SEQ ID NO:15 wherein Xaa at position 1 is the N-terminal residue of the C-protein and is therefore a splicing residue such as Ser, Cys, or Thr, for example having the following sequence (SEQ ID NO:13):

| | |
|---|---|
| Xaa Xaa Xaa Xaa Xaa Xaa Xaa Xaa Xaa Xaa Xaa Xaa Xaa Xaa Xaa | 15 |
| Xaa Xaa Xaa Xaa Xaa Xaa Xaa Xaa Xaa Xaa Xaa Xaa Xaa Xaa Xaa | 30 |
| Xaa Xaa Xaa Xaa Xaa Xaa Xaa Xaa Xaa Xaa Xaa Xaa Xaa Xaa Xaa | 45 |
| Xaa Xaa Xaa Xaa Xaa Xaa Xaa Xaa Xaa Xaa Xaa Xaa Xaa Xaa Xaa | 60 |
| Xaa Xaa Xaa Xaa Xaa Xaa Xaa Xaa Xaa Xaa Xaa Xaa Xaa Xaa Xaa | 75 |
| Xaa Xaa Xaa Xaa Xaa Xaa Xaa, | |

wherein:

Xaa at position 1 is a splicing residue such as Ser, Cys, or Thr;

Xaa at positions 2 to 39 can be any amino acid (e.g. Ala or Gly) or absent;

Xaa at position 40 is an amino acid suitable for conjugation to a water-soluble polymer, such as Cys, Lys, His, Arg, Asp, Glu, Ser, Thr, Tyr, or a ketone-containing unnatural amino acid such as para-acetyl-phenylalanine (pAcF), homocysteine, or selenocysteine; and

Xaa at positions 41 to 82 can be any amino acid (e.g. Ala or Gly) or absent.

In embodiments, one or more of Xaa residues at positions 41 to 82 of SEQ ID NO:13 (e.g. the carboxy-terminal residue) are chosen to inhibit proteolysis by enzymes found in human serum or plasma or blood.

In embodiments, at least one Xaa at positions 2 to 39 or 41 to 82 of SEQ ID NO:13 is an amino acid suitable for conjugation to a water-soluble polymer, as described above, thereby providing at least one additional site for attaching a water-soluble molecule to the carrier molecule.

Thus in embodiments, the C-extein of the C-protein can comprise a carrier molecule comprising the following sequence (SEQ ID NO:14):

Xaa Xaa Xaa Xaa Xaa Xaa Xaa

wherein:

Xaa at position 1 is a C-extein splicing residue such as Ser, Cys, or Thr;

Xaa at positions 2 to 4 can be any natural amino acid (e.g. Phe, Tyr, Trp, Ala or Gly);

Xaa at position 5 is an amino acid suitable for conjugation to a water-soluble polymer, such as Cys, Lys, His, Arg, Asp, Glu, Ser, Thr, Tyr, or a ketone-containing unnatural amino acid such as para-acetyl-phenylalanine (pAcF), homocysteine, or selenocysteine; and

Xaa at positions 6 and 7 can be any amino acid (e.g. Ala or Gly).

When Xaa at position 7 is the C-terminal residue of the N-protein, it is preferably a proteolysis-inhibiting amino acid such as Pro.

Specific examples of such C-exteins comprising carrier molecules therefore include: SGGGCGP (SEQ ID NO:11) and SAGGCGP (SEQ ID NO:12), wherein the serine residue is the C-extein splicing residue.

### E. Protein Trans-Splicing

The trans-splicing reaction is carried out by contacting said target polypeptide trans-splicing partner with its carrier molecule trans-splicing partner, under conditions suitable to induce excision of the split intein segments and joining of the exteins, thereby producing a compound that comprises the target polypeptide linked to the carrier molecule. The trans-splicing reaction can conveniently be carried out at room temperature (e.g. between about 15° C. to 30° C., preferably between about 20° C. to 25° C.) in an aqueous buffer (for example: 20 mM Tris-HCl or phosphate buffer, pH 8.0, 150 mM sodium chloride, 1 mM DTT or TCEP, 1 mM EDTA). The trans-splicing partners react in stochiometric amounts (a ratio of 1:1). However, yield may be improved by using of an excess of one of the trans-splicing partners (e.g. a ratio of about 2:1, about 3:1, about 4:1, about 5:1, about 6:1, about 7:1, about 8:1, about 9:1, about 10:1, about 20:1, about 50:1, or about 100:1). Therefore, in many cases, the reaction will be carried out using an excess of the carrier molecule trans-splicing partner, either before or after attachment of the water-soluble polymer molecule.

The splicing reaction produces a product having a target polypeptide linked to the carrier molecule. When the target polypeptide and carrier molecule were derived by splitting the sequence of a single polypeptide of interest, the spliced product will re-form the complete polypeptide of interest together with any few amino acid residues that may have been added to facilitate splicing. The region linking these the target polypeptide and the carrier molecule will comprise any extein residue(s) that flanked the I_{N} and I_{C} split intein components as may have been included to facilitate intein activity or to facilitate conjugation of the water soluble polymer. Thus, the product of the trans-splicing reaction will contain features that are characteristic of the trans-splicing partners, and may be identifiable. For example, when the method utilizes trans-splicing partners comprising SEQ ID NO:6 and SEQ ID NO:9, the linkage forms a peptide bond between the N-extein's C-terminal glycine and the C-extein's N-terminal serine, providing a linking sequence GS and a GSG sequence in the region linking the target polypeptide and carrier molecule. Similarly, when the method utilizes trans-splicing partners comprising SEQ ID NO:8 and SEQ ID NO:10, the linkage forms a peptide bond between the N-extein's C-terminal glycine and the C-extein's N-terminal serine, providing a linking sequence GS and a GSG sequence in the region linking the target polypeptide and carrier molecule. The rest of the extein portions are present in their entirety, and in the examples described herein, have the carrier molecule sequence SGGCGP (SEQ ID NO:11) as their new C-terminal sequence for SB trans-splicing and SAGGCGP (SEQ ID NO:12) as their new C-terminal sequence for the SG trans-splicing. The carrier molecule may either be already attached to a water-soluble polymer molecule or will allow the specific attachment of the polymer to it after splicing. In the examples shown, the polypeptide sequence was PEGylated before splicing. Depending on the number and state of any cysteine residues in the N-extein, the carrier molecule of the present examples may be used to provide the only available unpaired cysteine and thus allow highly specific PEGylation after splicing.

The polymer molecule(s) can be attached to the carrier molecule either before or after the trans-splicing reaction. However, it will be appreciated that, if the reaction conditions to be used for attaching the polymer molecule to the carrier molecule can cause the target polypeptide to lose activity (for example due to unfolding or attachment of the polymer to competing sites present on the target polypeptide), then it may be preferable to attach the polymer to the carrier molecule portion of the corresponding trans-splicing partner prior to the trans-splicing reaction, and optionally purify the resulting product prior to trans-splicing it to the target polypeptide trans-splicing partner. Alternatively, the polymer molecule(s) can be attached to the carrier molecule after the trans-splicing reaction, e.g. in cases where the attachment chemistry does not substantially interfere with the activity of the target polypeptide or where the activity of the target polypeptide can be restored by re-folding.

### F. Water-Soluble Polymers:

Suitable water-soluble polymer molecules for practicing the invention include but are not limited to: poly(ethylene glycol) (PEG); poly(ethylene glycol)monomethylether (MPEG); ethylene glycol/propylene glycol copolymers, carboxymethylcellulose, dextran, and polyvinyl alcohol. Other water-soluble polymer molecules that may be suitable for practicing the invention are described in U.S. Pat. No. 7,230,068 and EP0714402. As is known to one skilled in the art of PEGylation, both branched and linear polymer molecules are useful. The branched polymers may have two, three or more polymer segments joined to one another by a variety of known chemical methods, however, they generally comprise only one reactive site for coupling to the carrier peptide to prevent them from cross linking carrier peptide segments either before or after splicing. For example, see recent U.S. Pat. No. 7,291,713 for examples and references relating to branched PEG materials.

Water-soluble polymer molecules can be attached to target sites using any suitable method, and a variety of such methods are known, for example:

(a) Methods to PEGylate the amino-terminus of a target polypeptide are described for example in: U.S. Pat. No. 6,077,939; U.S. Pat. No. 7,090,835; U.S. Pat. No. 5,621,039; and Gilmore J M, Scheck R A, Esser-Kahn A P, Joshi N S, Francis M B. N-terminal protein modification through a biomimetic transamination reaction. Angew Chem Int Ed Engl. (2006) 45 5307-11.

(b) Methods to PEGylate an unpaired cysteine residue of a target polypeptide are described for example in U.S. Pat. No. 7,214,779 and Doherty D H, et al. Site-specific PEGylation of Engineered Cysteine Analogues of Recombinant Human Granulocyte-Macrophage Colony-Sytimluating Factor. Bioconjugate Chem. (2005) 16, 1291-1298).

(c) Methods to PEGylate an unnatural amino acid residue in a target polypeptide are described for example in U.S. Pat. No. 7,230,068.

(d) Methods to PEGylate an arginine group in a peptide or protein are disclosed in U.S. Pat. No. 5,093,531 (expired). Other 1,2 diketones and ketoaldehyde derivatives of water soluble polymers can be utilized to derivatize arginine residues (Pande, C. S. I M. Pelzig and J. D, Glass. “Camphorquinone-10-sulfonic acid and derivatives: convenient reagents for reversible modification of arginine residues.” Proc Natl Acad Sci USA. (1980) 77 895-9).

(e) U.S. Pat. No. 6,552,170 to Thompson provides many references to the current art of protein PEGylation and the various reactive derivatives that have been shown to be useful in coupling water soluble polymers to proteins or peptides. Any of these cited methods may be useful for PEGylating a carrier molecule that has been designed to have only one to three reactive sites. U.S. Pat. No. 6,010,999 to Daley utilizes the type of MPEG iodoacetamide reagents utilized in the examples herein to form MPEG conjugates by coupling to any of the several cysteins present in a specific protein. The '170 patent describes the formation of thioether bonds to cysteine residues of a target molecule by reagents different from those as are used in the examples herein and in the '999 patent and provides other thioether bond forming reagents that can be used as alternatives to the MPEG iodoacetamide utilized herein.

In exemplary embodiments, the carrier molecule contains one or more Cys residues as target sites for attaching polymer and, when the N-terminal residue of the carrier molecule is the splicing residue, an N-terminal Ser residue.

Examples of approaches that can be used to attach the polymer to the carrier molecule include but are not limited to:

(a) Reaction of an extein sulfhydryl group with a polymer maleimide group.

(b) Reaction of an extein sulfhydryl group with a polymer iodoacetamide group.

(c) Reaction of an extein sulfhydryl group with a polymer vinyl sulfone group.

(d) Reduction of an extein disulfide bond followed by reaction with one or both sulfhydryl groups with a polymer maleimide or iodoacetamide group.

(e) Reductive amination of the N-terminal group of the N-extein with a polymer aldehyde derivative to give a N-terminally derivatized N-extein.

(f) Oxidation of an N-terminal serine, threonine or cysteine with periodate such as disclosed in U.S. Pat. No. 5,821,343 to Keogh or published by Gaertner and Offord (Gaertner H F, Offord R E. “Site-specific attachment of functionalized polyethylene glycol) to the amino terminus of proteins.” Bioconjug Chem. (1996) 7(1):38-44) to form an N-terminal aldehyde containing N-Extein and conjugation with a water soluble polymer amine derivative via reductive amination.

(g) Oxidation of an N-terminal serine, threonine or cysteine with periodate such as disclosed in U.S. Pat. No. 5,821,343 to Keogh to form an N-terminal aldehyde containing N-Extein and conjugation with a water soluble polymer amine having an amide linked cysteine terminal group capable of forming a thiazolidine with the aldehyde.

(h) Oxidation of an N-terminal serine, threonine or cysteine carrier molecule with periodate such as disclosed in U.S. Pat. No. 5,821,343 to Keogh to form an N-terminal aldehyde containing N-Extein and conjugation with a water soluble polymer hydrazide, oxyamine or other aldehyde specific polymer reagent.

(i) Synthesis of an extein fragment capable of undergoing trans-splicing where the terminus of the extein has a reactive chemical functionality not normally found in proteins or peptides such as a ketone or halide or conjugated double bond or vicinyl diol such that the product can be directly or after further reaction to activate a reactive group precursor such as:
(i) with the oxidation of a diol to yield an aldehyde, or
(ii) the removal of an acid labile protective group to form a reactive aromatic amine, and
(iii) reactions that form covalent bonds specifically with the functional group normally not present in proteins and a specifically prepared polymer reagent capable of reacting with such an extein in a specific and selective manner.

(j) Coupling reactions such as the reaction with an azide and an acetylene derivative and other such reactions commonly referred to as Click Chemistry reactions (Kolb H C, Finn M G, Sharpless K B., “Click Chemistry: Diverse Chemical Function from a Few Good Reactions.”, Angew Chem Int Ed Engl. (2001), 40, 2004-2021. See also a recent review: Moses J E, Moorhouse A D. “The growing applications of click chemistry.” Chem Soc Rev. (2007) 36, 1249-62).

(k) The use of a split intein-related fragment capable of splicing and having an extein containing from zero to 2 lysine residues wherein the intein portion of said fragment does not contain a lysine residue and thus the relatively non-specific acylation of the amino groups of the fragment can be used to prepare a polymer conjugate with a specific number of attached polymer chains. Such reagents will usually react with the N-terminal amine and any lysine amine side chains to add from 1 to 3 chains, depending on the count of the amino groups present. The selectivity arises in the design of the intein-extein trans-splicing partner and accordingly it may be convenient to use a small extein to subsequently label the target protein or peptide. See U.S. Pat. No. 6,930,086 to Tischer for an example of a non-intein mediated coupling of a PEGylated polypeptide to a second polypeptide such that the product represented a reconstituted EPO molecule having one or two molecules of water soluble polymer attached.

The examples herein utilize the well established selectivity of MPEG maleimide or MPEG iodoacetamide for the sulfhydryl group of the cysteine incorporated into the extein as one example of a PEGylation method with high specificity and selectivity. Only mono-PEGylated reagent was observed via this chemistry. The split inteins of the present examples are particularly suited for this approach since they do not contain any cysteine residues in the C-Intein and the splicing junction is a serine on the PEGylated C-Extein. The use of an N-terminal serine or threonine or cysteine residue at the terminus of the N-terminal carrier molecule is an important embodiment that allows the selective and specific N-terminal PEGylation of a protein or peptide by reaction with a PEG aldehyde derivative. The use of an N-terminal carrier molecule with a single cysteine or lysine residue in the sequence for attachment of a water soluble polymer is another embodiment of the invention. The use of a serine at the N terminus of the C-extein and the inclusion of a cysteine in the C-extein carrier molecule is an embodiment of the invention.

The invention is further illustrated by the following non-limiting examples, which describe particular embodiments of the invention.

### EXAMPLES

In the following examples, these abbreviations are used: Dalton (Da); Diisopropylethylamine (DIEA); evaporative light scattering detector (ELSD); equivalents (Eq); kiloDalton or 1,000 Daltons (kDa); methanol (MeOH); 2-(N-morpholino)ethane sulfonic acid (MES); poly(ethylene glycol) monomethyl ether (MPEG); milli-Seimen (mS); ammonium acetate (NH4OAc); poly(ethylene glycol) (PEG); refractive index detector (RI); Size exclusion chromatography (SEC); triethylamine (TEA); tetrahydrofuran (THF); microlitre (microL); deionized water (DI water); sodium sulphate (Na₂SO₄); potassium bromide (KBr); and potassium hydroxide (KOH).

In addition, in the following examples:

SBsplit I_{N} (residues 2 to 103 of SEQ ID NO:6, or residues 397 to 498 of SEQ ID NO:2) is the N-intein (I_{N}) of engineered split SBnative N-terminal piece;

SBsplit I_{C} (residues 1 to 49 of SEQ ID NO:9, or residues 398 to 446 of SEQ ID NO:1) is the C-intein (I_{C}) of engineered split SBnative;

SGsplit I_{N} (residues 2 to 112 of SEQ ID NO:8, or residues 394 to 504 of SEQ ID NO:4) is the N-intein (I_{N}) of engineered split SGnative intein; and

SGsplit I_{C} (residues 1 to 45 of SEQ ID NO:10, or residues 398 to 442 of SEQ ID NO:3) is the C-intein (I_{C}) of engineered split SGnative intein.

### Example 1

### Production of the SB C-Protein Precursor (SEQ ID NO:1) of the SB C-Protein Trans-Splicing Partner

The native Ssp DnaB intein (SEQ ID NO:5) requires a serine as the splicing residue located at the N-terminal of the C-extein (Wu, H.; Xu, M. Q.; Liu, X. Q. (1998) Protein trans-splicing and functional mini-inteins of a cyanobacterial dnaB intein. Biochim Biophys Acta 1387:422-32.), which is unlike many other inteins that require a cysteine at that position. By taking advantage of this fact, we used the Ssp DnaB intein (SEQ ID NO:5) to produce a C-terminal trans-splicing partner that contains a single cysteine for site-specific PEGylation before or after trans-splicing onto the C-terminus of a target protein. To produce this polypeptide, we first constructed a fusion protein that is named SB C-protein precursor and schematically illustrated in FIG. 1B. This fusion protein consisted of a maltose binding protein (MBP), a Factor Xa cleavage site, a His-tag (6 histidine residues), SBsplit I_{C} (residues 1 to 49 of SEQ ID NO:9), which are very closely related to the last 49 residues of the native SspDnaB intein as reported by Wu et. al. (vide supra), and a peptide sequence SGGGCGP (SEQ ID NO:11) containing a single cysteine for PEGylation. The amino acid sequence of this fusion protein, and its corresponding nucleic acid coding sequence are shown in FIGS. 6A and 6B as SEQ ID NO:1 and SEQ ID NO:22, respectively.

The role of the MBP is as a supporting protein, to facilitate the protein production and/or purification. A Factor Xa protease cleavage site is present between the MBP and the His-tag, which allows the MBP to be removed before doing trans-splicing, as shown later in Example 3. The His-tag can be used to do a metal affinity chromatography purification of the fusion protein or the trans-splicing C-terminal polypeptide after the MBP has been removed. The split intein segment, SBsplit I_{C} is followed by a 7-residues sequence SGGGCGP (SEQ ID NO:11) to be spliced onto the C-terminus of a target protein, in which S (serine) is the splicing residue required for the trans-splicing reaction, C (cysteine) is for site-specific PEGylation, G (glycine) residues are to provide some spaces around the cysteine, and P (proline) is thought to minimize degradation by carboxyl peptidases.

The SB C-protein precursor was produced routinely as a recombinant protein in E. coli by cloning the protein coding sequence into a recombinant plasmid vector (pMST) behind an IPTG-inducible promoter (Wu et. al. (1998) Biochem Biophys Acta 1387:422-32, vide supra). The resulting expression plasmid was introduced into E. coli strain DH5α by using a standard electroporation method. The resulting transformed E. coli cells were grown in liquid LB medium to mid-log phase and induced with 0.8 mM IPTG to express the recombinant protein either at 37° C. for 3 hours or at room temperature for overnight. The cells were harvested by centrifugation and lysed by passing through a French Press Cell. The recombinant protein in the cell lysate was purified using routine techniques by using a metal affinity chromatography specific for the His-tag (Ni-NTA from Qiagen) or an amylose affinity chromatography (amylose resin from New England Biolabs) specific for the MBP by following the manufacturer's instructions for using these chromatography materials.

### Example 2

### Production of the SB N-Protein (SEQ ID NO:2) Trans-Splicing Partner

To complement the above SB C-protein for trans-splicing, we produced an N-terminal trans-splicing partner that is named SB N-protein (SEQ ID NO:2) and illustrated in FIG. 1B. This recombinant fusion protein consisted of a MBP (residues 1 to 383) and SBsplit I_{N} (residues 397 to 498 of SEQ ID NO:2). The amino acid sequence of this fusion protein and its corresponding nucleic acid coding sequence are shown in FIGS. 7A and 7B as SEQ ID NO:2 and SEQ ID NO:23, respectively.

The MBP part serves as a model target protein for PEGylation and also facilitates an amylose affinity purification of the fusion protein. The SBsplit I_{N} intein part has a peptide sequence closely related to the first 102 residues of the native Ssp DnaB Intein (Wu et. al. vide supra).

The SB N-protein was produced routinely as a recombinant protein in E. coli by cloning the protein coding sequence into a recombinant plasmid vector (pMST) behind an IPTG-inducible promoter (Wu et. al. (1998) Biochem Biophys Acta 1387:422-32, vide supra). The resulting expression plasmid was introduced into E. coli strain DH5α by using a standard electroporation method. The resulting transformed E. coli cells were grown in liquid LB medium to mid-log phase and induced with 0.8 mM IPTG to express the recombinant protein either at 37° C. for 3 hours or at room temperature for overnight. The cells were harvested by centrifugation and lysed by passing through a French Press Cell. The recombinant protein in the cell lysate was purified routinely by using a metal affinity chromatography on an amylose affinity chromatography (amylose resin from New England Biolabs) specific for the MBP by following the manufacturer's instructions.

### Example 3

### Production of the SG C-Protein Precursor (SEQ ID NO:3) of the SG C-Protein Trans-Splicing Partner

To demonstrate the trans-splicing PEGylation using a second and different intein, we constructed a fusion protein that is named SG C-protein precursor (SEQ ID NO:3) and illustrated in FIG. 1B. The amino acid sequence of this fusion protein and its corresponding nucleic acid coding sequence are shown in FIGS. 8A and 8B as SEQ ID NO:3 and SEQ ID NO:24, respectively.

With the following exceptions, this SG C-protein precursor is otherwise identical to the SB C-protein precursor described in Example 1. The split intein component is SGsplit I_{C} (residues 1 to 45 of SEQ ID NO:10), followed by a 7-residues sequence SAGGCGP (SEQ ID NO:12) to be spliced onto the C-terminus of a target protein, in which S (serine) is required for the trans-splicing reaction, C (cysteine) is for site-specific PEGylation, G (glycine) and A (alanine) residues are to provide some spaces around the cysteine, and P (proline) is thought to minimize degradation by carboxyl peptidases. The intein part has a peptide sequence closely related to the last 45 residues of the native Ssp GyrB Intein (Dalgaard, J. Z.; Moser, M. J.; Hughey, R.; Mian, I. S. “Statistical modeling, phylogenetic analysis and structure prediction of a protein splicing domain common to inteins and hedgehog proteins.” J Comput Biol (1997) 4:193-214.)

The expression and purification of this SG C-protein precursor were carried out in the same way as described above for the SB C-protein precursor in Example 1.

### Example 4

### Production of the SG N-Protein (SEQ ID NO:4) Trans-Splicing Partner

To complement the above SG C-protein for trans-splicing, we produced a trans-splicing N-terminal protein that is named SG N-protein (SEQ ID NO:4) and illustrated in FIG. 1B. The amino acid sequence of this fusion protein and its corresponding nucleic acid coding sequence are shown in FIGS. 9A and 9B as SEQ ID NO:4 and SEQ ID NO:25, respectively.

With the following exceptions, this SG N-protein is otherwise identical to the SB N-protein described in Example 2. The split intein part is SGsplit I_{N} (residues 2 to 112 of SEQ ID NO:8), immediately preceded by GG, compared to LRESG (SEQ ID NO:21) in the SB N-protein. The intein part has a peptide sequence closely related to the first 111 residues of the native Ssp DnaB I_{N} Intein as reported in Dalgaard et al. (Dalgaard, J. Z.; Moser, M. J.; Hughey, R.; Mian, I. S. “Statistical modeling, phylogenetic analysis and structure prediction of a protein splicing domain common to inteins and hedgehog proteins.” J Comput Biol (1997) 4:193-214.)

The expression and purification of this SG N-protein were carried out in the same way as described above for the SB N-protein in Example 2.

### Example 5

### PEGylation of the SB C-Protein Precursor

Materials used for PEGylation of proteins: Sodium Phosphate Monobasic (NaH2PO4), Sodium hydroxide (NaOH), Ethylenediaminetetraacetic acid (EDTA) disodium salt dihydrate, Guanidine hydrochloride were purchased from Sigma. Argon was purchased from Canada Air Liquid. Tris(2-carboxyethyl)phosphine hydrochloride (TCEP), Dithiothreitol (DTT), Vectra™ MPEG iodoacetamide 20 kDa were products of BioVectra DCL. The SB C-protein precursor (SEQ ID NO:1) from Example 1 was buffer exchanged with argon saturated 0.1M phosphate buffer, pH 8.3, containing 3 M guanidine and 2 mM EDTA on 5 kDa MWCO membrane centrifugal filter (Millipore™), and concentrated to 0.1 mL. To this solution was added 0.01 mL of 0.1 M TCEP or DTT. This mixture was incubated at ambient for 30-120 min under argon atmosphere followed by gel filtration (Bio-Spin® 6 Tris Column: BioRad laboratories) using a mini-column which was equilibrated with 0.1M phosphate buffer, pH 8.3, containing 3 M guanidine and 2.0 mM EDTA. The high molecular weight fraction was collected into a tube containing 1.4 mg Vectra MPEG iodoacetamide 20 kDa (BioVectra DCL). This reaction mixture was incubated at ambient temperature for 12-48 hours.

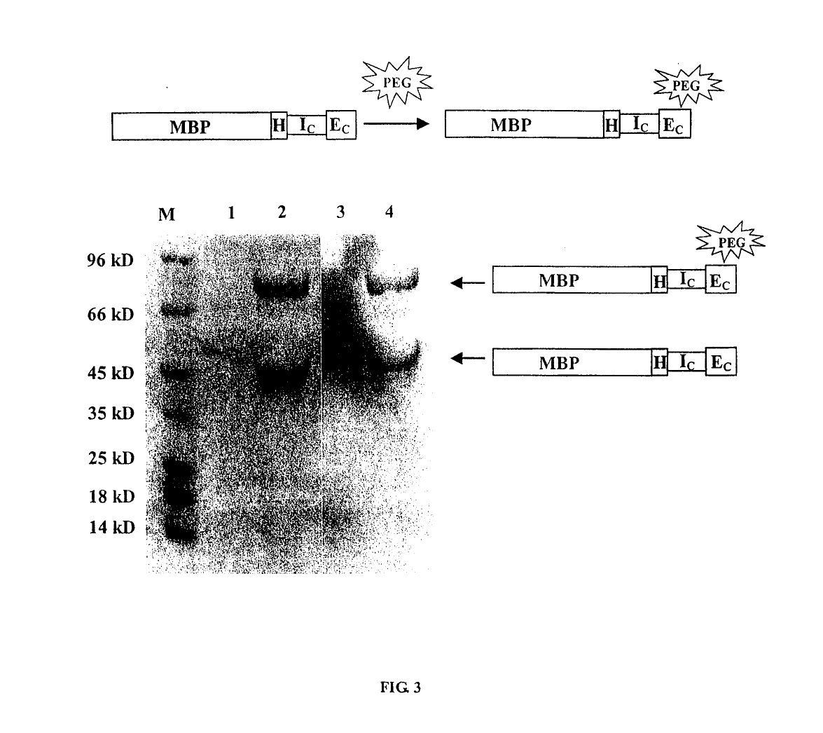

Results of the PEGylation were analyzed by SDS-PAGE and are shown in FIG. 3. Successful PEGylation of the SB C-protein precursor (SEQ ID NO:1) was indicated by its conversion into a PEGylated form that showed a much larger size. Based on the protein band intensity following electrophoresis, more than 50% of the SB C-protein precursor amount was converted into the PEGylated form.

### Example 6

### PEGylation of the SG C-Protein Precursor (SEQ ID NO:3)

The SG C-protein precursor (SEQ ID NO:3) from Example 3 (0.5 mL, 1 mg) was buffer exchanged with argon saturated 0.1M phosphate buffer, pH 8.3, containing 3 M guanidine and 2 mM EDTA on 5 kDa MWCO membrane centrifugal filter (Millipore), and concentrated to 0.1 mL. To this solution added was 0.01 mL of 0.1 M TECP or DTT. This mixture was incubated at ambient for 30-120 min under argon atmosphere followed by gel filtration (Bio-Spin® 6 Tris Column: BioRad laboratories) using media that was equilibrated with 0.1M phosphate buffer, pH 8.3, containing 3 M guanidine and 2.0 mM EDTA. The high molecular weight fraction was collected into a tube containing 1.4 mg Vectra MPEG-iodoacetamide 20,000 Da (BioVectra DCL). This reaction mixture was incubated at ambient for 12-48 hours.

Results of the PEGylation was analyzed by SDS-PAGE and shown in FIG. 3. Successful PEGylation of the SG C-protein precursor was indicated by its conversion into a PEGylated form that showed a much larger size. Based on the protein band intensity following electrophoresis, more than 50% of the SG C-protein precursor amount was converted into the PEGylated form.

### Example 7

### Production of PEGylated SB C-Protein by Removing MBP from the PEGylated SB C-Protein Precursor (SEQ ID NO:1)

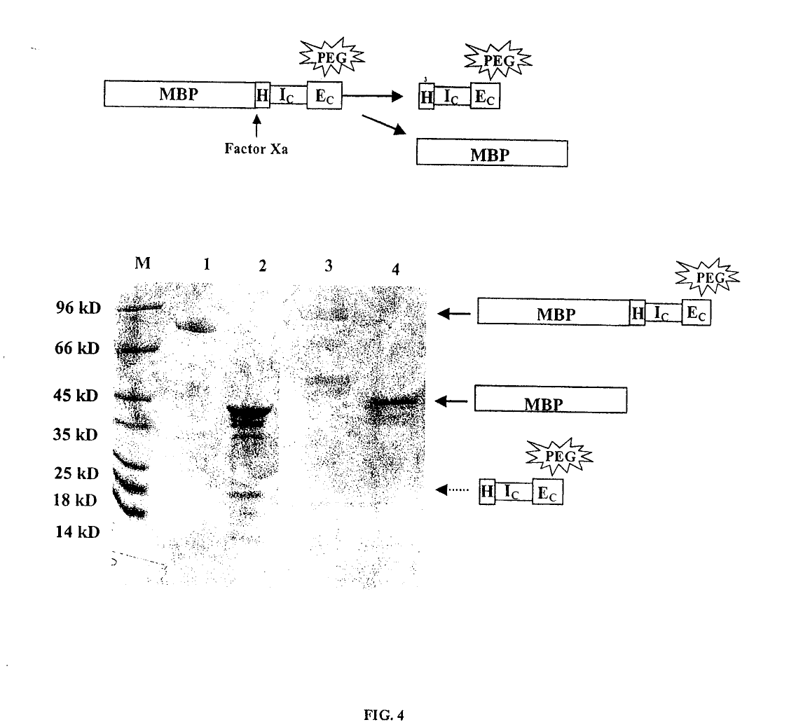

To produce the PEGylated SB C-protein (residues 388 to 453 of SEQ ID NO:1) for the trans-splicing, the large PEGylated SB C-protein precursor (SEQ ID NO:1) was treated with the Factor Xa protease to cleave off the MBP part. The PEGylated SB C-protein was dialyzed into a cleavage buffer (20 mM Tris-HCl, pH 8.0, 1M NaCl, 20 mM CaCl₂). To every 100 mg of the protein, 1 mg Factor Xa (New England Biolabs) was added, and the mixture was incubated at 4° C. overnight to allow the cleavage to occur. The cleavage results were analyzed by SDS-PAGE and shown in FIG. 4. The results showed a successful and complete cleavage, as indicated by the complete disappearance of the SB C-protein precursor (both PEGylated form and unPEGylated form which did not stain with the Coomassie blue staining protocol used) and the appearance of the released MBP. The released SB C-protein (both PEGylated form and unPEGylated form) could not be seen by the method used, presumably because this short peptide could not be visualized by Coomassie blue staining.

To purify the SB C-protein away from the released MBP and the Factor Xa protease, the above cleavage products were passed through a metal affinity column. Only the SB C-protein contained the His-tag, therefore could bind to the column, and could be eluted in a pure form, after the MBP and Factor Xa protease (both lacked the His-tag) had been washed off the column. The column was prepared by pouring 2 ml of the Ni-NTA slurry (Qiagen™) in a 0.8×4 cm column, followed by washing the column with 5 volumes of the wash buffer (20 mM Tris-HCl, pH 8.0, 1M NaCl). The cleavage products were loaded onto the column at a flow rate of 1 ml/minute. After washing the column with 10 volumes of the wash buffer, the SB C-protein was eluted with 250 mM imidazole in the wash buffer.

### Example 8

### Production of PEGylated SG C-Protein by Removing MBP from the PEGylated SG C-Protein Precursor (SEQ ID NO:3)

The small PEGylated SG C-protein (residues 388 to 449 of SEQ ID NO:3) was prepared from the large PEGylated SG C-protein precursor (SEQ ID NO:3) and purified away from the MBP and the Factor Xa protease, in exactly the same way as for the PEGylated SB C-protein described in Example 7.

### Example 9

### Trans-Splicing of the PEGylated SB C-Protein with the SB N-Protein

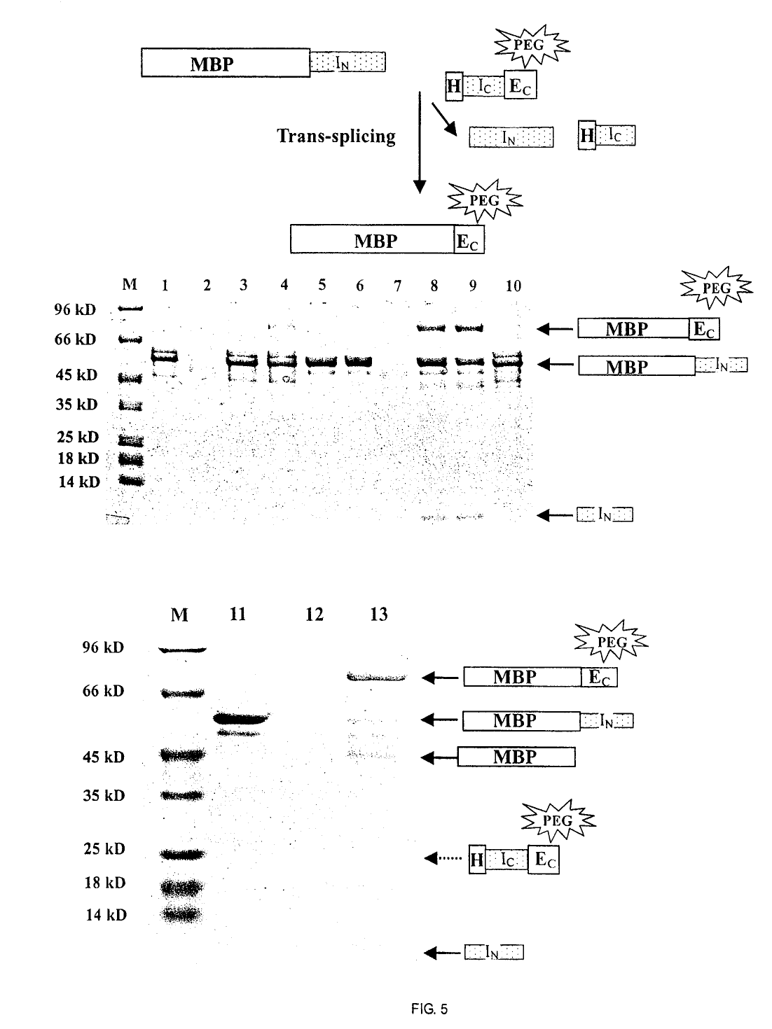

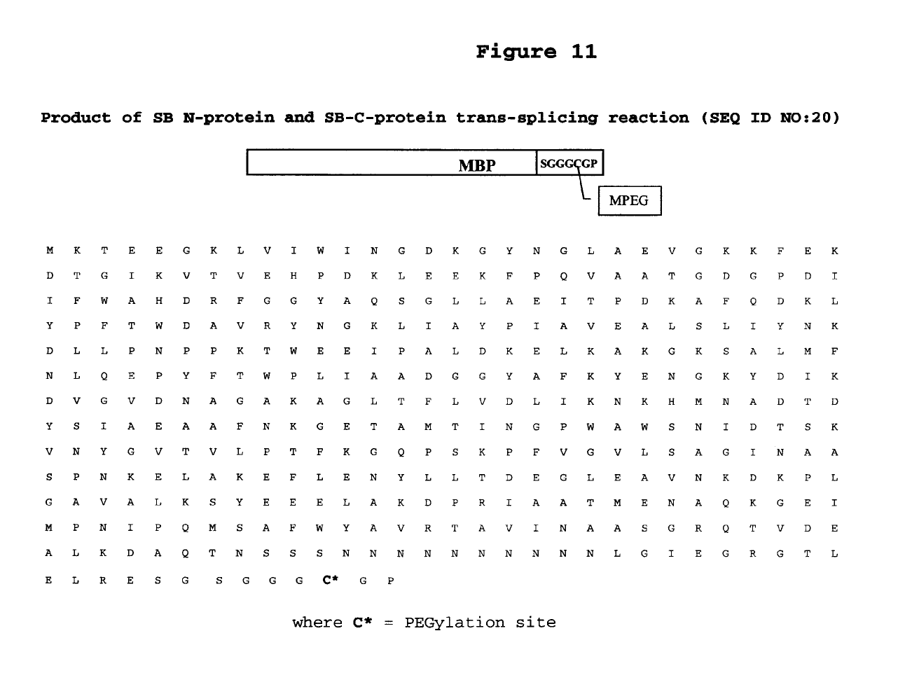

To carry out a trans-splicing reaction using the PEGylated SB C-protein from Example 7, the SB C-protein was incubated with the SB N-protein from Example 2, with the C-protein to the N-protein molar ratio being approximately 1. The incubation was in a trans-splicing buffer (20 mM Tris-HCl, pH 8.0, 150 mM NaCl, 1 mM DTT, 1 mM EDTA) either at room temperature for 3 hours or at 4° C. overnight. The results were analyzed by SDS-PAGE and are shown in FIG. 5 (lanes 1-4). Successful trans-splicing was observed after incubation at room temperature, but not at 4° C., as indicated by the appearance of a new and larger protein band corresponding to the expected trans-spliced product. The efficiency of the trans-splicing reaction was estimated at ˜30%, based on the protein band intensity. The product of ligating the exteins is shown in SEQ ID NO:20 (FIG. 11).

### Example 10

### Trans-Splicing the PEGylated SG C-Protein with the SG N-Protein

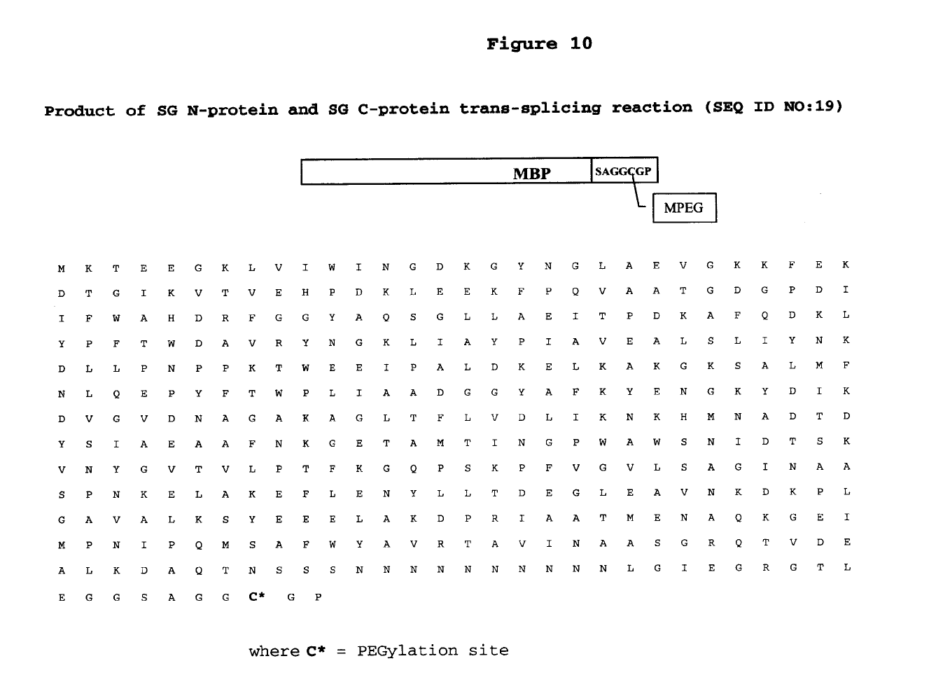

To carry out a trans-splicing reaction using the PEGylated SG C-protein from Example 8, the peptide was incubated with the SG N-protein from Example 4. The incubation was in a trans-splicing buffer (20 mM Tris-HCl, pH 8.0, 150 mM NaCl, 1 mM DTT, 1 mM EDTA) either at room temperature for 3 hours or at 4° C. overnight, and the molar ratio of the C-protein to the N-protein was initially approximately 1. The results were analyzed by SDS-PAGE and are shown in FIG. 5. Successful trans-splicing was observed after incubation both at room temperature (Lane 9) and at 4° C. (Lane 8), as indicated by the appearance of a new and larger protein band corresponding to the expected trans-spliced product. The efficiency of the trans-splicing was estimated at ˜50% (Lane 9), based on the protein band intensity. This efficiency increased to approximately 90% (Lane 13) when the molar ratio of the C-protein to the N-protein was increased to approximately 5. The product of ligating the exteins is shown in SEQ ID NO:19 (FIG. 10).

Unless defined otherwise all technical and scientific terms used herein have the same meaning as commonly understood to one of ordinary skill in the art to which this invention belongs.

All publications and patent applications cited in this specification are herein incorporated by reference as if each individual publication or patent application were specifically and individually indicated to be incorporated by reference.

The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of modifying a target polypeptide, comprising:
(a) providing a first trans-splicing partner which comprises a first component of a split intein in operative linkage with a first extein segment, wherein the first extein comprises at least one functional group suitable for attaching at least one water-soluble polymer molecule;
(b) providing a second trans-splicing partner which comprises a second component of the split intein in operative linkage with a second extein segment that comprises the target polypeptide, wherein said first and second trans-splicing partners are capable of cooperating to provide protein trans-splicing (PTS) activity; and
(c) contacting said first and second trans-splicing partners under conditions suitable to induce excision of the first and second components of the split intein and joining of the extein segments, so as to ligate the first extein to the second extein,
wherein the at least one water-soluble polymer is attached to the first extein either before or after the first extein is ligated to the second extein.

2. The method of claim 1, wherein the water-soluble polymer is attached to the first extein before the first extein is ligated to the target polypeptide of the second extein.

3. The method of claim 1, wherein the split intein is selected from:
(a) a split intein comprising SBsplit I_{N} (residues 2 to 102 of SEQ ID NO:6) and SBsplit I_{C} (residues 1 to 49 of SEQ ID NO:9), or a functional variant thereof;
(b) a split intein comprising SGsplit I_{N} (residues 2 to 111 of SEQ ID NO:8) and SGsplit I_{C} (residues 1 to 45 of SEQ ID NO:10), or a functional variant thereof;
(c) a split intein from the DnaE gene of Synechocystis sp. PCC6803, or a functional variant thereof;
(d) a cyanobacterial dnaB split intein, or a functional variant thereof;
(e) an artificially split Ssp DnaB intein, or a functional variant thereof;
(f) an artificially split Sce VMA intein, or a functional variant thereof;
(g) an artificially split fungal mini-intein, or a functional variant thereof; and
(h) Npu DanE split intein, or a functional variant thereof.

4. The method of claim 1, wherein the first component of the split intein is a split intein N-terminal component (I_{N}) and the second component of the intein is a split intein C-terminal component (I_{C}).

5. The method of claim 4, wherein:
(a) the first trans-splicing partner comprises the amino acid sequence set forth in SEQ ID NO:6, such that the I_{N} has the amino acid sequence set forth in residues 2 to 102 of SEQ ID NO:6 and the C-terminal residue of the first extein is Gly; and
(b) the second trans-splicing partner comprises the amino acid sequence set forth in SEQ ID NO:9, such that the I_{C} has the amino acid sequence set forth in residues 1 to 49 of SEQ ID NO:9 and the N-terminal residues of the second extein are Ser-Gly.

6. The method of claim 4, wherein:
(a) the first trans-splicing partner comprises the amino acid sequence set forth in SEQ ID NO:8, such that the I_{N} has the amino acid sequence set forth in residues 2 to 111 of SEQ ID NO:8 and the C-terminal residue of the first extein is Gly; and
(b) the second trans-splicing partner comprises the amino acid sequence set forth in SEQ ID NO:10, such that the IC has the amino acid sequence set forth in residues 1 to 45 of SEQ ID NO:10 and the N-terminal residues of the second extein are Ser-Ala.

7. The method of claim 5, wherein the first extein comprises an amino acid sequence as set forth in SEQ ID NO:16.

8. The method of claim 1, wherein the first component of the split intein is a split intein C-terminal component (I_{C}) and the second component of the split intein is a split intein N-terminal component (I_{N}).

9. The method of claim 8, wherein:
(a) the second trans-splicing partner comprises the amino acid sequence set forth in SEQ ID NO:6, such that the I_{N} has the amino acid sequence set forth in residues 2 to 102 of SEQ ID NO:6 and the C-terminal residue of the first extein is Gly; and
(b) the first trans-splicing partner comprises the amino acid sequence set forth in SEQ ID NO:9, such that the I_{C} has the amino acid sequence set forth in residues 1 to 49 of SEQ ID NO:9 and the N-terminal residues of the second extein are Ser-Gly.

10. The method of claim 8, wherein:
(a) the second trans-splicing partner comprises the amino acid sequence set forth in SEQ ID NO:8, such that the I_{N} has the amino acid sequence set forth in residues 2 to 111 of SEQ ID NO:8 and the C-terminal residue of the first extein is Gly; and
(b) the first trans-splicing partner comprises the amino acid sequence set forth in SEQ ID NO:10, such that the I_{C} has the amino acid sequence set forth in residues 1 to 45 of SEQ ID NO:10 and the N-terminal residues of the second extein are Ser-Ala.

11. The method of claim 9, wherein the second extein comprises an amino acid sequence as set forth in SEQ ID NO:11.

12. The method of claim 10, wherein the second extein comprises an amino acid sequence as set forth in SEQ ID NO:12.

13. The method of claim 1, wherein the first extein comprises a Cys residue and the water-soluble polymer molecule is attached to the sulfhydryl group of said Cys residue.

14. The method of claim 1, wherein the water-soluble polymer molecule is poly(ethylene glycol) (PEG).

15. The method of claim 1, wherein the water-soluble polymer molecule is poly(ethylene glycol) monomethyl ether (MPEG).

16. A chemically modified polypeptide produced by the method of claim 1.

17. The chemically modified polypeptide of claim 16, wherein the first extein comprises a Cys residue and the water-soluble polymer molecule is attached to the sulfhydryl group of said Cys residue.

18. The chemically modified polypeptide of claim 16, wherein the water-soluble polymer molecule is poly(ethylene glycol) (PEG).

19. The chemically modified polypeptide of claim 16, wherein the water-soluble polymer molecule is poly(ethylene glycol) monomethyl ether (MPEG).

20. A polypeptide comprising:
(a) an N-terminal or C-terminal component of a split intein; and
(b) an extein segment that comprises at least one functional group suitable for attaching at least one water-soluble polymer molecule wherein the extein segment is in operative linkage with the split intein component, or a conjugate thereof which is covalently bonded to said water-soluble polymer.

21. The polypeptide of claim 20, or the conjugate thereof, which comprises amino acid residues 388 to 453 or 398 to 453 of SEQ ID NO:1.

22. The polypeptide of claim 20, or the conjugate thereof, which comprises amino acid residues 388 to 449 or 398 to 449 of SEQ ID NO:3.

23. The polypeptide of claim 20, wherein the water-soluble polymer molecule is poly(ethylene glycol) (PEG).

24. The polypeptide of claim 20, wherein the water-soluble polymer molecule is poly(ethylene glycol) monomethyl ether (MPEG).

25. A nucleic acid molecule encoding the polypeptide of claim 20.

26. A vector comprising the nucleic acid of claim 25 in operative linkage with a promoter.

27. A host cell comprising the vector of claim 26.

28. A method of producing a polypeptide comprising:
(a) an N-terminal or C-terminal component of a split intein; and
(b) an extein segment that comprises at least one functional group suitable for attaching at least one water-soluble polymer molecule wherein the extein segment is in operative linkage with the split intein component, or a conjugate thereof which is covalently bonded to said water-soluble polymer, said method comprising culturing the host cell of claim 27 under conditions suitable to induce expression of said polypeptide.

29. A polypeptide comprising an N-terminal component of a split intein, wherein the polypeptide comprises the amino acid sequence as set forth in SEQ ID NO:6, or a variant thereof having at least 50% identity thereto and that is capable of interacting with a complementary C-terminal component of the split intein to provide trans-splicing activity.

30. A polypeptide comprising a C-terminal component of a split intein, wherein the polypeptide comprises the amino acid sequence as set forth in SEQ ID NO:9, or a variant thereof having at least 50% identity thereto and that is capable of interacting with a complementary N-terminal component of the split intein to provide trans-splicing activity.

31. A polypeptide comprising an N-terminal component of a split intein, wherein the polypeptide comprises the amino acid sequence as set forth in SEQ ID NO:8, or a variant thereof having at least 50% identity thereto and that is capable of interacting with a complementary C-terminal component of the split intein to provide trans-splicing activity.

32. A polypeptide comprising a C-terminal component of a split intein, wherein the polypeptide comprises the amino acid sequence as set forth in SEQ ID NO:10, or a variant thereof having at least 50% identity thereto and that is capable of interacting with a complementary N-terminal component of the split intein to provide trans-splicing activity.

33. A nucleic acid encoding the polypeptide of claim 29.

34. A vector comprising the nucleic acid of claim 33 in operative linkage with a promoter.

35. A host cell comprising the vector of claim 34.

36. A method of producing a polypeptide, comprising culturing the host cell of claim 35 under conditions suitable to induce expression of said polypeptide.

37. Use of the polypeptide according to claim 36 in a protein trans-splicing reaction.


SEQUENCE LISTING (25 sequences)

SEQ ID NO: 1 - protein, 453 aa, Artificial sequence
  amino acid sequence for SB C-protein precursor
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRGTLEHHHHHHGSPEIEKLSQSDIYWDPIVSITETGVEEVFDLTVPGPRNFVANDIIVHNSGGGCGP

SEQ ID NO: 2 - protein, 498 aa, Artificial sequence
  amino acid sequence for SB N-protein
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRGTLELRESGCISGDSLISLASTGKRVSIKDLLDEKDFEIWAINEQTMKLESAKVSRVFCTGKKLVYILKTRLGRTIKATANHRFLTIDGWKRLDELSLKEHIALPRKLEGA

SEQ ID NO: 3 - protein, 449 aa, Artificial sequence
  amino acid sequence for SG C-protein precursor
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRGTLEHHHHHHGSEAVLNYNHRIVNIEAVSETIDVYDIEVPHTHNFALASGVFVHNSAGGCGP

SEQ ID NO: 4 - protein, 504 aa, Artificial sequence
  amino acid sequence for SG N-protein
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRGTLEGGCFSGDTLVALTDGRSVSFEQLVEEEKQGKQNFCYTIRHDGSIGVEKIINARKTKTNAKVIKVTLDNGESIICTPDHKFMLRDGSYKCAMDLTLDDSLMPLHRKISTTEDSG

SEQ ID NO: 5 - protein, 432 aa, Synechocystis sp.
GCISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLMSDEELGLLGHLIGDGCTLPRHAIQYTSNKIELAEKVVELAKAVFGDQINPRISQERQWYQVYIPASYRLTHNKKNPITKWLENLDVFGLRSYEKFVPNQVFEQPQRAIAIFLRHLWSTDGCVKLIVEKSSRPVAYYATSSEKLAKDVQSLLLKLGINARLSKISQNGKGRDNYHVTITGQADLQIFVDQIGAVDKDKQASVEEIKTHIAQHQANTNRDVIPKQIWKTYVLPQIQIKGITTRDLQMRLGNAYCGTALYKHNLSRERAAKIATITQSPEIEKLSQSDIYWDSIVSITETGVEEVFDLTVPGPHNFVANDIIVHNSI

SEQ ID NO: 6 - protein, 103 aa, Artificial sequence
  SBsplit N-intein has the amino acid sequence of residues 2 to 103
GCISGDSLISLASTGKRVSIKDLLDEKDFEIWAINEQTMKLESAKVSRVFCTGKKLVYILKTRLGRTIKATANHRFLTIDGWKRLDELSLKEHIALPRKLEGA

SEQ ID NO: 7 - protein, 438 aa, Synechocystis sp.
GCFSGDTLVALTDGRSVSFEQLVEEEKQGKQNFCYTIRHDGSIGVEKIINARKTKTNAKVIKVTLDNGESIICTPDHKFMLRDGSYKCAMDLTLDDSLMPLHRKISTTEDSGITIDGYEMVWSPRSDSWLFTHLVADWYNRWQGIYIAEEKQHCHHKDFNKRNNNPDNLIRLSPEKHLALHRKHISKTLHRPDVVEKCRRIHQSPEFRRKMSARMQSPETRAILSKQAQAQWQNETYKLTMMESWRSFYDSNEDYRQQNAEQLNRAQQEYWAQAENRTAQAERVRQHFAQNPGLRQQYSENAVKQWNNPELLKWRQKKTKEQWTPEFREKRREALAQTYYRKTLAALKQVEIENGYLDISAYDSYRISTKDKSLLRFDRFCERYFENDENLAREAVLNYNHRIVNIEAVSETIDVYDIEVPHTHNFALASGVFVHNSA

SEQ ID NO: 8 - protein, 112 aa, Artificial sequence
  SGsplit N-intein has the amino acid sequence of residues 2 to 112
GCFSGDTLVALTDGRSVSFEQLVEEEKQGKQNFCYTIRHDGSIGVEKIINARKTKTNAKVIKVTLDNGESIICTPDHKFMLRDGSYKCAMDLTLDDSLMPLHRKISTTEDSG

SEQ ID NO: 9 - protein, 51 aa, Artificial sequence
  SBsplit C-intein has the amino acid sequence of residues 1 to 49
GSPEIEKLSQSDIYWDPIVSITETGVEEVFDLTVPGPRNFVANDIIVHNSG

SEQ ID NO: 10 - protein, 47 aa, Artificial sequence
  SGsplit C-intein has the amino acid sequence of residues 1 to 45
GSEAVLNYNHRIVNIEAVSETIDVYDIEVPHTHNFALASGVFVHNSA

SEQ ID NO: 11 - protein, 7 aa, Artificial sequence
  peptide carrier molecule
SGGGCGP

SEQ ID NO: 12 - protein, 7 aa, Artificial sequence
  peptide carrier molecule
SAGGCGP

SEQ ID NO: 13 - protein, 82 aa, Artificial sequence
  polypeptide carrier molecule
XXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 14 - protein, 7 aa, Artificial sequence
  polypeptide carrier molecule
XXXXXXX

SEQ ID NO: 15 - protein, 82 aa, Artificial sequence; identical to SEQ ID NO: 13
  polypeptide carrier molecule

SEQ ID NO: 16 - protein, 6 aa, Artificial sequence
  polypeptide carrier molecule
PGCGGG

SEQ ID NO: 17 - protein, 6 aa, Artificial sequence
  polypeptide carrier molecule
PGCGGA

SEQ ID NO: 18 - protein, 6 aa, Artificial sequence
  polypeptide carrier molecule
XXXXXX

SEQ ID NO: 19 - protein, 400 aa, Artificial sequence
  product of SG N-protein and SG C-protein trans-splicing reaction
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRGTLEGGSAGGCGP

SEQ ID NO: 20 - protein, 403 aa, Artificial sequence
  product of SB N-protein and SB C-protein trans-splicing reaction
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRGTLELRESGSGGGCGP

SEQ ID NO: 21 - protein, 5 aa, Artificial sequence
  peptide
LRESG

SEQ ID NO: 22 - DNA, 1362 bp, Artificial sequence
  nucleic acid sequence for SB C-protein precursor
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggggtacgctcgagcaccatcatcaccaccatggatccccagaaatagaaaagttgtctcagagtgatatttactgggaccccatcgtttctattacggagactggagtcgaagaggtttttgatttgactgtgccaggaccacgtaactttgtcgccaatgacatcattgtccataactcaggtggcggttgtggtccgtaa

SEQ ID NO: 23 - DNA, 1497 bp, Artificial sequence
  nucleic acid sequence for SB N-protein
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggggtacgctcgagttaagagagagtggctgcatcagtggagatagtttgatcagcttggcgagcacaggaaaaagagtttctattaaagatttgttagatgaaaaagattttgaaatatgggcaattaatgaacagacgatgaagctagaatcagctaaagttagtcgtgtattttgtactggcaaaaagctagtttatattctaaaaactcgactaggtagaactatcaaggcaacagcaaatcatagatttttaactattgatggttggaaaagattagatgagctatctttaaaagagcatattgctctaccccgtaaactagaaggcgcctga

SEQ ID NO: 24 - DNA, 1350 bp, Artificial sequence
  nucleic acid sequence for SG C-protein precursor
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggggtacgctcgagcaccatcatcaccaccatggatccgaagcagtattaaattacaatcacagaattgtaaatattgaagctgtgtcagaaacaatcgatgtttatgatattgaggttccccacacccacaattttgctttggcaagcggagtgtttgtccataacagcgctggcggttgtggtccgtaa

SEQ ID NO: 25 - DNA, 1515 bp, Artificial sequence
  nucleic acid sequence for SG N-protein
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggggtacgctcgagggcggttgtttttctggagatacattagtcgctttaactgatggtcgtagcgttagctttgagcaattggttgaagaagaaaaacaaggaaaacaaaacttttgttataccatccgccatgatggttctataggggttgaaaaaatcatcaatgcccgcaaaacaaaaactaatgcgaaggtaatcaaggttacgttggacaatggtgagtctattatttgcaccccggatcataaattcatgttgcgggatgggagctacaaatgtgcgatggatttaactctcgatgattcgttaatgccgttacaccgaaaaatttcgactacggaagattctggttaa